(19) United States
(12) Patent Application Publication
(10) Pub. No.: US 2023/0391831 A1
(43) Pub. Date: Dec. 7, 2023

(54) FORMULATION FOR WOUND HEALING

(71) Applicant: ARIZONA BOARD OF REGENTS ON BEHALF OF ARIZONA STATE UNIVERSITY, Scottsdale, AZ (US)

(72) Inventors: Alexandra Lucas, Tempe, AZ (US); Liqiang Zhang, Chandler, AZ (US); Jordan Yaron, Scottsdale, AZ (US)

(21) Appl. No.: 18/032,080

(22) PCT Filed: Oct. 13, 2021
(86) PCT No.: PCT/US2021/054834

§ 371 Date: Apr. 14, 2023

Related U.S. Application Data
(60) Provisional application No. 63/092,330, filed on Oct. 15, 2020.

(51) Int. Cl. C07K 14/005; A61K 45/06; A61K 9/00; A61P 17/02

(52) U.S. Cl. CPC C07K 14/005; A61K 45/06; A61K 9/0014; A61P 17/02; A61K 38/00

(57) ABSTRACT

Wound healing is a process by which these wounds on the skin of a subject heal and eventually close. When the injured surface is large, becomes infected, or in patients with poor healing capacity such as diabetics or the elderly or bedridden patients, then wound healing can be prolonged and lead to chronic ulceration and further complications with even limb loss or increased morbidity and mortality. This invention is directed to a topical formulation for promoting wound healing, and wound dressings and bandages comprising the same.

This application claims priority from U.S. Provisional Application No. 63/092,330, filed on Oct. 15, 2020, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to a topical formulation for promoting wound healing, and wound dressings and bandages comprising the same.

### BACKGROUND OF THE INVENTION

Wounds (i.e., lacerations or openings) in mammalian tissue result in tissue disruption and coagulation of the microvasculature at the wound face. Wound healing is a process by which these wounds on the skin of a subject heal and eventually close. Repair of such tissue represents an orderly, controlled cellular response to injury. Soft tissue wounds, regardless of size, heal in a similar manner. Tissue regrowth and repair are biologic systems wherein cellular proliferation and angiogenesis occur in the presence of an oxygen gradient. The sequential morphological and structural changes which occur during tissue repair have been characterized in great detail and have in some instances been quantified. When the injured surface is large, becomes infected, or in patients with poor healing capacity such as diabetics or the elderly or bedridden patients, then wound healing can be prolonged and lead to chronic ulceration and further complications with even limb loss or increased morbidity and mortality.

### SUMMARY OF THE INVENTION

Aspects of the invention are drawn to a topical formulation for promoting wound healing. For example, the topical formulation comprises a therapeutically effective amount of an M-T7 polypeptide.

In certain embodiments, a therapeutically effective amount is between about 0.1 μg/kg and about 100 mg/kg. For example, the therapeutically effective amount is about 0.1 μg/kg, about 1 μg/kg, about 10 μg/kg, about 100 μg/kg, about 1 mg/kg, about 10 mg/kg, or about 100 mg/kg. In embodiments, the therapeutically effective amount is greater than about 100 mg/kg.

In some embodiments, a therapeutically effective amount is between about 0.01 mg/ml and about 500 mg/ml. For example, a therapeutically effective amount is about 0.01 mg/ml, about 0.1 mg/ml, about 1 mg/ml, about 10 mg/ml, about 100 mg/ml, about 200 mg/ml, about 300 mg/ml, about 400 mg/ml, about 500 mg/ml, or greater than 500 mg/ml.

In embodiments, the topical formulation further comprises a pharmaceutically acceptable carrier.

In embodiments, the topical formulation comprises a biologically active fragment of M-T7 derived from Myxoma-virus.

In embodiments, the M-T7 polypeptide is recombinantly produced.

In embodiments, the M-T7 polypeptide has an amino acid sequence at least 80% identical to the amino acid sequence of

(SEQ ID NO: 1)
MDGRLVFLLASLAIVSDAVRLTSYDLNTFVTWQDDGYTYNVSIK
PYTTATWINVCEWASSSCNVSLALQYDLDVVSWARLTRVGKYTE
YSLEPTCAVARFSPPEVQLVRTGTSVEVLVRHPVVYLRGQEVSV
YGHSFCDYDFGYKTIFLFSKNKRAEYVVPGRYCDNVECRFSIDS
QESVCATAVLTYGDSYRSEAGVEVCVPELAKREVSPYIVKKSSD
LEYVKRAIHNEYRLDTSSEGRRLEELYLTVASMFERLVEDVFE

In embodiments, the M-T7 polypeptide has an amino acid sequence at least 80% identical to the amino acid sequence of

(SEQ ID NO: 2)
VRLTSYDLNTFVTWQDDGYTYNVSIKPYTTATWINVCEWASSSC
NVSLALQYDLDVVSWARLTRVGKYTEYSLEPTCAVARFSPPEVQ
LVRTGTSVEVLVRHPVVYLRGQEVSVYGHSFCDYDFGYKTIFLF
SKNKRAEYVVPGRYCDNVECRFSIDSQESVCATAVLTYGDSYRS
EAGVEVCVPELAKREVSPYIVKKSSDLEYVKRAIHNEYRLDTSS
EGRRLEELYLTVASMFERLVEDVFE

In embodiments, the M-T7 polypeptide lacks the N-terminus secretion sequence.

In embodiments, the M-T7 polypeptide has a minimal amino acid sequence at least 80% identical to a polypeptide fragment within SEQ ID NO: 1 or SEQ ID NO: 2.

In embodiments, the topical formulation further comprises one or more additional active ingredients. For example, the one or more additional active ingredients comprises an antibiotic, a pain reliever, an anti-inflammatory, an anti-scarring agent, a moisturizer, a steroid, an immune modulator, or a growth factor.

In embodiments, the topical formulation is contained in a hydrophilic polymer. For example, the hydrophilic polymer comprises a hydrogel. For example, the hydrogel comprises chitosan, collagen, glycerine, aloe vera, methyl paraben, hydrogenated castor oil, hyaluronic acid, polypeptides, pHEMA, pHPMA, or any combination thereof.

In embodiments, the M-T7 polypeptide comprises one or more post-translational modifications.

In embodiments, the M-T7 polypeptide comprises one or more modifications to a post-translational modification.

For example, the post-translational modification is selected from the group consisting of PEGylation, sialylated, glycosylation, acetylation, acylation, lipid modification, palmitoylation, palmitate addition, phosphorylation, or glycolipid modification.

In embodiments, the M-T7 polypeptide comprises at least one mutation. For example, the mutation comprises F(137)D, R(171)E, and/or E(209)I.

In embodiments, the composition is formulated as a topical ointment, cream, lotion, suspension, aqueous solution, dispersion, salve, gel, spray, or paste.

Aspects of the invention are also drawn to a nucleic acid encoding the M-T7 polypeptide, such as an M-T7 polypeptide described herein. In embodiments, the nucleic acid has a nucleic acid sequence at least 80% identical to the nucleic acid encoding the M-T7 polypeptide. In embodiments, the nucleic acid is a component of a vector. In embodiments, the vector is in a cell.

Still further, aspects of the invention are drawn to a wound dressing or bandage. For example, the wound dressing or bandage comprises a therapeutically effective amount of an M-T7 polypeptide as described herein. For example, the M-T7 polypeptide is in a formulation that is added to, coated, on, or embedded into the wound dressing or bandage.

Further, aspects of the invention are drawn to a method of treating a wound in a subject in need thereof. For example, the wound is a dermal wound, a chronic wound, an infected wound, a burn wound, a diabetic wound, a skin wound, or a cutaneous wound.

In embodiments, the method comprises administering topically onto the wound the topical formulation as described herein, wherein the formulation comprises an M-T7 polypeptide. In other embodiments, the method comprises applying onto the wound of a subject a wound dressing or bandage as described herein.

Further, aspects of the invention are drawn to a method of promoting angiogenesis in a subject in need thereof. In embodiments, the method comprises administering topically onto the wound the topical formulation as described herein, wherein the formulation comprises an M-T7 polypeptide. In other embodiments, the method comprises applying onto the wound of a subject a wound dressing or bandage as described herein. .

Also, aspects of the invention are drawn to a kit comprising the topical formulation as described herein.

Further, aspects of the invention are drawn to methods of accelerating wound healing by application of a chemokine modulating protein, such as M-T7 or a polypeptide thereof. For example, treatment with M-T7 provides a method to decrease damaging inflammation by altering chemokine to glycosamino glycan binding and improving angiogenesis.

Aspects also provide compositions and methods for improved nervous system wound healing.

Other objects and advantages of this invention will become readily apparent from the ensuing description.

### BRIEF DESCRIPTION OF THE FIGURES

FIG. 1 shows the M-T7 accelerates full-thickness wound healing in mice. (A) Experimental design overview. Mice were wounded on Day 0 (green arrow) and followed to Day 15 post-wounding (red arrow), at which time mice were euthanized and tissue was collected. Mice were treated with saline or M-T7 on Day 0 with a second bolus give on Day 3 post-wounding (blue arrow). Mice were anesthetized and splints were removed on Day 7 post-wounding (yellow arrow). Mice were assessed daily and images were collected for planimetric measurement of wound closure (gray arrows). (B) Planimetric measurements of wound closure normalized to Day 0 for each mouse. Mean and standard error are shown. Statistics were calculated by T-test per-day with correction for multiple comparisons by the Holm-Sidak method. For significance, a equates to p<0.05, b equates to p<0.01 and c equates to p<0.001. (C) Representative wound images for saline and M-T7-treated mice on the day of wounding (Day 0) and on Days 2, 4, 7 and 15 post-wounding. The same mouse is shown in each image per condition. Scale bars are 5 mm.

FIG. 2 shows quantitative assessment of collagen maturation in wounds treated with M-T7. (A) Representative micrographs of Herovici's polychrome-stained normal skin and wounds at 15 days post-wounding. Top panels show brightfield data, while middle and bottom panels show color-deconvoluted fields for the pink and blue chromophores. (B) Quantification of collagen maturation in saline and M-T7 treated wounds by the Herovici Ratio, calculated by the densitometric ratio of the pink and blue chromophores in Herovici's polychrome. Mean and standard error are shown. Statistics were calculated by T-test. N=4 saline, N=5 M-T7.

FIG. 3 shows assessment of peri-wound angiogenesis in wounds treated with M-T7. (A) ELISA quantification of TNFα and VEGF in wound tissues treated with saline or M-T7 collected on days 1, 4 and 7 post-wounding normalized to total protein. Bars are mean and standard error. Statistics were calculated by two-way ANOVA with Fisher's LSD post-hoc analysis. (B) Quantification of CD31+cells and vessels per 20× field in the peri-wound area of wounds treated with saline or M-T7 collected on days 4 and 7 post-wounding. Bars are mean and standard error. Two non-overlapping fields were quantified per mouse and statistics were performed on the average per mouse with the N=4 per group. Statistics were calculated by two-way ANOVA with Fisher's LSD post-hoc analysis. (C) Representative peri-wound CD31 IHC fields (10×) collected on days 4 and 7 post-wounding. Scale bars are 50 μm. Zoom areas indicated by boxes. N=3-4 in each group and time point.

FIG. 4 shows M-T7 modulates the immune response in the healing wound. (A) ELISA quantification of CCL2 in wounds treated with saline or M-T7 at days 1, 4 and 7 post-wounding, normalized to total protein. (B) Quantification of Arginase-1+ cells per 20× field of wounds treated with saline or M-T7 at days 2, 4 and 7 post-wounding. (C) Representative Arginase-1 IHC fields at day 7. (D) Quantification of TGF-beta+cells per 20× field on days 2, 4 and 7 post-wounding. (E,F) Quantification of CD3+ cells per 20× field of wounds treated with saline or M-T7 at days 2, 4 and 7 post-wounding, specifically in the (E) wound bed or (F) epithelial tongue. (G) Quantification of CD4+ cells per 20× field of wounds treated with saline or M-T7 at days 2, 4 and 7 post-wounding normalized to the numbers on day 2. (H) Representative CD4 IHC fields in the epithelial tongue at day 7. Full 20× field is given in FIG. 6. All bars are mean and standard error. Statistics are calculated by two-way ANOVA with Fisher's LSD post-hoc analysis. N=3-4 in each group and time point.

FIG. 5 shows quantification of IHC staining for HSP47+ cells per 20× field on tissues of mice on days 4 and 7 post-wounding and treated with saline or M-T7. Statistics analyzed by two-way ANOVA with Fisher's LSD post-hoc analysis. All bars are mean and standard error. N=3-4 mice per treatment per time point.

FIG. 6 shows full-frame 20× fields of CD4 IHC on Day 7 post-wounding for mice treated with saline and M-T7. Corresponds to FIG. 4, panel H in the main manuscript. Images are representative of 3-4 mice per treatment.

FIG. 7 shows quantification of IHC staining for Ly6G+ cells per 20× field on tissues of mice on days 4 and 7 post-wounding and treated with saline or M-T7. Statistics analyzed by two-way ANOVA with Fiscer's LSD post-hoc analysis.

FIG. 8 shows SDS-PAGE and Coomassie Blue staining demonstrating M-T7 cross linking. BS 3 : Thermo Scientific Pierce BS 3 (Sulfo-DSS) is bis(sulfosuccinimidyl)suberate; Reaction buffer: PBS, pH 7.8; Condition: BS3:M-T7=0-120 (molar ratio), on ice for 1 hours, then room temperature for 15min. Quenched by adding 1/15 volume of Tris-HC1 (1.0M, pH8.0).

FIG. 9 shows Ni-NTA resin purification of M-T7 from cell culture medium and western blot using anti-His Tag. M-T7 stable cell line: host cell is CHO-K1; carrier plasmid is pCMV. Full length of M-T7 with His9 tag is on C-terminal. Secreted expression

### DETAILED DESCRIPTION OF THE INVENTION

Large surface wounds, including lacerations and bums, are common and often complex injuries. In some cases, comorbidities such as diabetes and advanced age cause skin lesions to tum into non-healing chronic wounds, reducing function and increasing risk of infection and bleeding. Chronic non-healing wounds can be life threatening and are a major threat to public health and a large cost to the economy (Sen et al., Wound Repair Regen. 17 (2009) 763-71, Nussbaum et al., Value Health. 21 (2018) 27-32). According to the NIH ARRA Impact Report, over 6 million cases of chronic wounds occur annually in the United States with a collective cost of more than $20 billion per year. Severe burn injuries cause about 40,000 hospitalizations and nearly 4,000 deaths each year. Notably, these numbers do not include scar revisions, which amount to over 170,000 procedures annually in the USA (Lim et al., Plast. Reconstr. Surg. 133 (2014) 398-405).

The wound healing process is frequently divided into three steps: hemostasis and inflammation, new tissue generation and remodeling (Eming et al., Sci. Transl. Med.6 (2014)), where the immune system plays a central role in each step. Wound healing in adults can begin with bleeding and clot formation (haemostasis) followed by a rapid-onset of inflammation. Immune response cells, including neutrophils (Wilgus et al., Adv. Wound Care. (2013), Soehnlein et al., Nat. Rev. Immunol. (2017)) and macrophages (Lucas et al., J. Immunol. 184 (2010) 3964-3977, Hesketh et al., Int. J. Mol. Sci. (2017), Wynn and Vannella, Immunity. 44 (2016) 450-462, Mantovani et al., J. Pathol. (2013), Brancato and AlbinaAm. J. Pathol. 178 (2011) 19-25) are known to be crucial in initiating the early stage of wound healing.

Acute inflammation is critical to healthy wound healing, with innate immunity driving early responses to injury and with precisely regulated stages at both the cellular and molecular levels, while sustained and excessive inflammation can exacerbate damage and result in chronic wounds (Eming et al., J. Invest. Dermatol. (2007), Landen et al., Cell. Mol. Life Sci. (2016))

It has been widely recognized that modulating the immune system through biomaterials and drug delivery systems can alter wound healing, increasing regeneration and reducing fibrosis (Zhao etal., Int. J. Mol. Sci. (2016); Julier et al., Acta Biomater. (2017); Stejskalova and Almquist, Biomater. Sci. 5 (2017) 1421-1434). The three major factors that are known to fundamentally alter wound healing and management are infection, wound closure and fibrosis (scarring). Accordingly, attention has been directed towards technologies that inhibit infection, promote wound closure, and reduce scarring, for example, individually or simultaneously. Wound healing in chronic or infected wounds is a major health problem causing morbidity and increasingmortality in Diabetic or burn patients.

Myxoma virus (MYXV) is a Leporipoxvirus with well-known strict species-specificity and host-tropism to the European rabbit (Oryctolagus cuniculus). The safety and immunotherapeutic efficacy of several MYXV immune modulators have been demonstrated in a wide array of preclinical models. M-T7 is a MYXV-derived immune modulator with broad C, CC and CXC class chemokine-binding activity and proven therapeutic activity in inflammation-related diseases. M-T7 is expressed early in MYXV infection and is the most abundantly secreted immune modulator.

As described herein, M-T7 polypeptide and fragments thereof are engineered and adapted as a new protein biologic for promoting wound healing. In a mouse wound healing model M-T7 significantly accelerates wound closure and also increases angiogrnesis. As such, aspects of the invention are drawn to formulations, such as topical formulations, for promoting wound healing, wherein the formulation comprises a therapeutically effective amount of an M-T7 polypeptide. Aspects of the invention are also drawn to a wound dressing or bandage comprising a therapeutically effective amount of an M-T7 polypeptide. For example, the wound dressing or bandage comprises the formulation described herein. Still further, aspects of the invention are drawn to methods of treating a wound in a subject, such as a chronic wound, a dermal wound, or an infected wound. Also, aspects of the invention are drawn to methods of promoting angiogenesis in a subject, such as to promote wound healing, by administering topically onto a wound a formulation as described herein.

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A Meyers (ed.), Molecular Biology and Biotechnology: a Comprehensive Desk Reference, published by VCH Publishers, Inc., 1995 (ISBN 9780471185710); and other similar references.

Suitable methods and materials for the practice or testing embodiments of the invention are described herein. Such methods and materials are illustrative only and are not intended to be limiting. Although methods and materials similar or equivalent to those described herein can be used in the practice or testing of embodiments of the invention, suitable methods and materials are described herein. All publications, patent applications, patents, and other references mentioned herein are incorporated by reference in their entirety.

Unless otherwise defined, all technical terms used herein have the same meaning as commonly understood. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999. In addition, the materials, methods, and examples are illustrative only and not intended to be limiting.

Wound Healing Formulations

Aspects of the invention are directed towards compositions and formulations for promoting wound healing.

A “formulation” can refer to a composition containing at least one active therapeutic agent or pharmaceutical, and one or more excipients. For example, a “topical formulation” can refer to a composition containing at least one active therapeutic agent or pharmaceutical, including an excipient, in which the therapeutic agent or pharmaceutical can be placed for direct application to a skin surface and from which an effective amount of therapeutic agent or pharmaceutical is released. Examples of topical formulations include but are not limited to ointment, cream, lotion, suspension, aqueous solution, dispersion, salve, gel, spray, or paste.

A “carrier,” “pharmaceutically acceptable carrier”, “excipient”, and the like can be used interchangeably, and can refer to any liquid, gel, paste, salve, solvent, liquid, diluent, fluid ointment base, suspension, spray, liposome, micelle, giant micelle, and the like, which is suitable for use in contact with living animal or human tissue without causing adverse physiological responses, and which does not interact with the other components of the composition in a deleterious manner. A number of carrier ingredients are known for use in making topical formulations, such as gelatin, polymers, fats and oils, lecithin, collagens, alcohols, water, etc

A “hydrogel” can refer to a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as alginate, chitosan, polyphosphazenes, and polyacrylates such as polyhydroxyethyl methacrylate (poly-HEMA) and poly-N-(2-hydroxypropyl) methacrylamide (poly-HPMA), which are crosslinked ionically, or block copolymers such as PLURONICSTM (BASF Corporation) or TETRONICSTM (BASF Corporation), polyethylene oxide-polypropylene glycol block copolymers which are crosslinked by temperature or pH sensing probes, respectively. Other materials include proteins such as fibrin, polymers such as polyvinylpyrrolidone, hyaluronic acid and collagen.

A “peptide”, “polypeptide”, and/or protein, which can be used interchangeably, can refer to any compound composed of amino acids, amino acid analogs, chemically bound together. Amino acids can be chemically bound together via amide linkages (CONH). Additionally, amino acids can be bound together by other chemical bonds. For example, the amino acids can be bound by amine linkages. Peptides include oligomers of amino acids, amino acid analog, or small and large peptides, including polypeptides or proteins.

Disclosed herein topical formulations that include M-T7 polypeptides and/or biologically active fragments and derivatives thereof, for example, M-T7 polypeptides and fragments thereof that promote wound healing in a mammalian subject topically administered a M-T7 polypeptide or a fragment thereof.

In certain embodiments, a topical formulation includes an effective amount, such as a therapeutically effective amount of a M-T7 polypeptide. An “effective amount” or “therapeutically effective amount” can refer to an amount of a compound or composition of this invention that is sufficient to produce an effect, which can be a therapeutic and/or beneficial effect. The effective amount will vary with the age, general condition of the subject, the severity of the condition being treated, the agent administered, the duration of the treatment, the nature of any concurrent treatment, the pharmaceutically acceptable carrier or excipient used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

In certain embodiments, a therapeutically effective amount is between about 0.1μg/kg and about 100 mg/kg. For example, the therapeutically effective amount is about 0.1 μg/kg, about 1 μg/kg, about 10 μg/kg, about 100 μg/kg, about 1 mg/kg, about 10 mg/kg, or about 100 mg/kg. In embodiments, the therapeutically effective amount is greater than about 100 mg/kg.

In some embodiments, a therapeutically effective amount is between about 0.01 mg/ml and about 500 mg/ml. For example, a therapeutically effective amount is about 0.01 mg/ml, about 0.1 mg/ml, about 1 mg/ml, about 10 mg/ml, about 100 mg/ml, about 200 mg/ml, about 300 mg/ml, about 400 mg/ml, about 500 mg/ml, or greater than 500 mg/ml.

In certain embodiments, a therapeutically effective amount of the formulation can be administered, such as topically, once a day, twice a day, three times a day, or as needed. In other embodiments, the therapeutically effective amount of the formulation can be administered every other day, every three days, once a week, or every other week, or monthly.

In certain embodiments, a M-T7 polypeptide has been modified so that splice sites are removed.

In embodiments, a M-T7 polypeptide comprises the amino acid sequence set forth as:

(SEQ ID NO: 1)
MDGRLVFLLASLAIVSDAVRLTSYDLNTFVTWQDDGYTYNVSIKPYTTA
TWINVCEWASSSCNVSLALQYDLDVVSWARLTRVGKYTEYSLEPTCAVA
RFSPPEVQLVRTGTSVEVLVRHPVVYLRGQEVSVYGHSFCDYDFGYKTI
FLFSKNKRAEYVVPGRYCDNVECRFSIDSQESVCATAVLTYGDSYRSEA
GVEVCVPELAKREVSPYIVKKSSDLEYVKRAIHNEYRLDTSSEGRRLEE
LYLTVASMFERLVEDVFE

In embodiments, the M-T7 polypeptide lacks the N-terminus secretion sequence. For example, in some embodiments, the M-T7 polypeptide comprises the amino acid sequence set forth as the sequence below, as predicted by SignalP5.0:

(SEQ ID NO: 2)
VRLTSYDLNTFVTWQDDGYTYNVSIKPYTTATWINVCEWASSSCNVSLA
LQYDLDVVSWARLTRVGKYTEYSLEPTCAVARFSPPEVQLVRTGTSVEV
LVRHPVVYLRGQEVSVYGHSFCDYDFGYKTIFLFSKNKRAEYVVPGRYC
DNVECRFSIDSQESVCATAVLTYGDSYRSEAGVEVCVPELAKREVSPYI
VKKSSDLEYVKRAIHNEYRLDTSSEGRRLEELYLTVASMFERLVEDVFE

M-T7 polypeptides includes polypeptides having at least 80%, such as at least 81%, at least 82%, at least 83%, at least 84%, at least 85%, at least 86%, at least 87%, at least 88%, or at least 89% at least 90%, at least 91%, at least 92%, at least 93%, at least 94%, at least 95%, at least 96%, at least 97%, at least 98%, at least 99% or even 100% sequence identity to the amino acid sequence set forth as SEQ ID NO: 1 and SEQ ID NO: 2, as well as biologically active fragments thereof. For example, biologically active fragments can include polypeptides of about 5, about 10, about 15, about 20, about 25, about 30, about 35, about 40, about 45, about 50, or greater than 50 amino acids.

The disclosed isolated peptides include synthetic embodiments of peptides described herein. In addition, analogs (non-peptide organic molecules), derivatives (chemically functionalized peptide molecules obtained starting with the disclosed peptide sequences), mutants, and variants (homologs) of these peptides can be utilized in the compositions ans methods described herein. Each peptide of this disclosure is comprised of a sequence of amino acids, which can be L- and/or D-amino acids, naturally occurring and otherwise. For example, the mutation can be F(137)D, R(171)E, and/or E(209)I. Such mutations, for example, can enhance or reduce M-T7 biologically activity.

Peptides can be modified by a variety of chemical techniques to produce derivatives having essentially the same activity as the unmodified peptides, and optionally having other desirable properties. In another example, carboxylic acid groups of the protein, whether carboxyl-terminal or side chain, can be provided in the form of a salt of a pharmaceutically-acceptable cation or esterified to form a C1-C1 6 ester, or converted to an amide of formula NR1R2 wherein R1 and R2 are each independently H or C1-C1 6 alkyl, or combined to form a heterocyclic ring, such as a 5- or 6-membered ring. Amino groups of the peptide, whether amino-terminal or side chain, can be in the form of a pharmaceutically-acceptable acid addition salt, such as the HC1, HBr, acetic, benzoic, toluene sulfonic, maleic, tartaric and other organic salts, or can be modified to C1-C1 6 alkyl or dialkyl amino or further converted to an amide.

Hydroxyl groups of the peptide side chains can be converted to C1-C1 6 alkoxy or to a C1-C1 6 ester using well-recognized techniques. Phenyl and phenolic rings of the peptide side chains can be substituted with one or more halogen atoms, such as fluorine, chlorine, bromine or iodine, or with C1-C1 6 alkyl, C1-C1 6 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides to select and provide conformational constraints to the structure that result in enhanced stability. While the peptides of the disclosure can be linear or cyclic, cyclic peptides can have an advantage over linear peptides in that their cyclic structure is more rigid and hence their biological activity can be higher than that of the corresponding linear peptide. Any method for cyclizing peptides can be applied to the serpin-derived peptides or fragments described herein.

In embodiments, the M-T7 polypeptide comprises one or more post-translational modifications or modifications there of. Examples of such post-translational modifications include, but are not limited to, glycosylation, deglycosylation, sialylation, acetylation, acylation, lipid modification, palmitoylation, palmitate addition, phosphorylation, glycolipid modification, PEGylation, methylation, and the like. In embodiments, the cell line that produces the M-T7 and/or the culture conditions can change the post-translational modification profile and activity of M-T7 polypeptide.

In embodiments, the peptide modification can be PEGylation, or linking of the M-T7 polypeptide to polyethylene glycol, so as to increase solubility and prolong circulatory time, for example. Once linked to a peptide, the PEG subunit becomes tightly associated with two or three water molecules, which has the dual function of rendering the polypeptide more soluble in water and making its molecular structure larger. As the kidneys filter substances according to size, the addition of PEG's molecular weight can prevent the premature renal clearance undergone by small peptides. PEG's globular structure can also act as a shield to protect the polypeptide of the invention from proteolytic degradation, and can reduce the immunogenicity of foreign peptides by limiting their uptake through the dendritic cells. PEG itself is not immunogenic or toxic, and allows for lower doses and less-frequent administrations. In some instances, PEG can increase the circulating half-life of a peptide drug by more than 100 times. In addition to improving the pharmacokinetic and pharmacodynamic properties of peptide drugs once inside the body, PEGylation can also aid drug delivery because PEGylated peptides act as permeation enhancers for nasal drug delivery.

In embodiments, the PEG molecule can be monomethoxy PEG (mPEG), which has relatively simple chemistry due to its monofunctionality (CH30-(CH2CH20)n-CH2CH2-OH). In other embodiments, the PEG molecule can be HiPEG, or PEG attached to histidine sequences expressed on the N or C terminal of proteins. For example, 6 His-tags can be used to create site-specific PEGylated conjugates, that is, PEGylation using a His-tagging approach. A protein is encoded with a polyhistidine tag (such as a 6 histidine tag). Once incubated with a Ni—nitrilotriacetic acid (NTA)-PEG reagent, a complex is formed between the histidine residues and the nickel ion, thus PEGylating the protein. In other embodiments, the PEG molecule can be branched or forked PEG, such as PEG2, releasable PEGs (rPEGs), or heterbifunctional PEGs, details of which can be found in Roberts, et al, which is incorporated by reference herein in its entirety (Roberts, M. J., M. D. Bentley, and J. M. Harris. “Chemistry for peptide and protein PEGylation.” Advanced drug delivery reviews 64 (2012): 116-127). One of ordinary skill in the art appreciates the routine methods practiced to pegylate amino acid residues of peptides of interest.

In embodiments, the peptide modification can be methylation. The methylation of proteins, for example, can help regulate cellular functions such as transcription, cell division, and cell differentiation. Methylation of the M-T7 polypeptide, for example, can extend the half-life of the peptides. Methylation of amino acid residues can be performed according to methods well understood by one of ordinary skill in the art (see US20090264620 and Mini Rev Med Chem. 2016; 16(9):683-90, each of which are incorporated by reference herein in their entireties entirety).

In embodiments, the peptide modification can be amidation or acetylation, such as at the C terminus or N terminus, respectively. Such modifications can also increase the metabolic stability of the peptides, as well as their ability to resist enzymatic degradation by aminopeptidases, exopeptidases, and synthetases. Amidation and acetylation of amino acid residues can be performed according to methods well understood by the skilled artisan, for example see Cottingham, Ian R., et al. “A method for the amidation of recombinant peptides expressed as intein fusion proteins in Escherichia coli.” Nature biotechnology 19.10 (2001): 974-977, Cerovsky, Vaclav, and Maria-Regina Kula. “Peptide amidase-catalyzed C-terminal peptide amidation in a mixture of organic solvents.” Peptides for the New Millennium (2002): 142-143; Mura, Manuela, et al. “The effect of amidation on the behaviour of antimicrobial peptides.” European Biophysics Journal 45.3 (2016): 195-207; Thomas, A., Towards a Functional Understanding of Protein N-Terminal Acetylation. PLOS Biol. 2011, 9(5); and Wallace, R. J., Acetylation of peptides inhibits their degradation by rumen micro-organisms. British Journal of Nutrition. 1992, 68, 365-372, each of which are incorporated by reference in their entireties.

In embodiments, the peptide modification can be acetylation, for example N-terminal acetylation (see U.S. Pat. No. 9,062,093, which is incorporated herein by reference in its entirety). This modification makes the resulting peptide more stable towards enzymatic degradation resulting from exopeptidases.

As noted, the M-T7 polypeptides can vary in length and can be or can include contiguous amino acid residues that naturally occur in M-T7, non-contiguous amino acids, or amino acids that vary to a certain degree from a naturally occurring M-T7 sequence (but retain a biological activity). Where the fragments include, at their N-terminus or C -terminus (or both), amino acid residues that are not naturally found in M-T7 the additional sequence(s), and can be about 200 amino acid residues long, and these residues can be divided evenly or unevenly between the N- and C-termini. For example, both the N- and C-termini can include about 10, 20, 30, 40, 50, 60, 70, 80, 90, or 100 amino acid residues.

Alternatively, one terminus can include about 10, 20, 30, 40, 50, 60, 70, 80, 90, 100, 110, 120, 130, 140, 20 150, 160, 170, 180, 190, or 200 residues, and one terminus can include none (e.g., it can terminate in an amino acid sequence identical to a naturally occurring M-T7 sequence).

More specifically, the N- or C-termini can include 1 to about 100 (e.g., 1, 2, 3, 4, 5, 6, 7, 8, 9, 10, 15, 20, 25, 30, 40, 50, 60, 70, 80, 90, or 100) amino acid residues that are positively charged (e.g., basic amino acid residues such as arginine, histidine, and/or lysine residues); 1 to about 100 amino acid residues that are negatively charged (e.g., acidic amino acid residues such as aspartic acid or glutamic acid residues); 1 to about 100 glycine residues; 1 to about 100 hydrophobic amino acid residues (e.g., hydrophobic aliphatic residues such as alanine, leucine, isoleucine or valine or hydrophobic aromatic residues such as phenylalanine, tryptophan or tyrosine); or 1 to about 100 (e.g., 1-4) cysteine residues. Where biologically active variants of a M-T7 fragment are used, the variant can vary by substitution of one or more amino acid residues within these groups. The variants can include a conservative amino acid substitution.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of a peptide having measurable M-T7 activity. For computer modeling applications, a pharmacophore is an idealized three-dimensional definition of the structural requirements for biological activity. Peptido- and organomimetics can be designed to fit each pharmacophore with current computer modeling software.

In embodiments, a M-T7 polypeptide is included in a fusion protein. Thus, the fusion protein can include a M-T7 polypeptide and a second heterologous moiety, such as a myc protein, an enzyme or a carrier (such as a hepatitis carrier protein or bovine serum albumin) covalently linked to the M-T7 polypeptide. A second heterologous moiety can be covalently or non-covalently linked to the Serp-1 polypeptide. The M-T7 polypeptide can be included in a fusion protein and can also include heterologous sequences.

In embodiments, the M-T7 polypeptide can be conjugated to a macromolecule, non-limiting examples of which comprise carrier proteins such as keyhole limpet hemocyanin (KLH), tetanus toxoid (TT), or bovine serum albumin (BSA). Conjugation of the peptide to such molecules, for example, can increase the stability of the peptide, or can increase resistance to proteolytic cleavage. Conjugation methods as listed herein are well understood by the skilled artisan (Chapter 3 Peptide-carrier conjugation: Laboratory Techniques in Biochemistry and Molecular Biology; Volume 19, 1988, Pages 95-130).

“Conjugation” can refer to the linking of a peptide, either directly or indirectly, to another molecule. For example, “direct conjugation” can refer to linking of the M-T7 polypeptide to an activated carbohydrate, another antigenic universal peptide, or a peptide linker, without introducing additional functional groups. As another example, “indirect conjugation” can refer to the addition of functional groups which are used to facilitate conjugation. For example, carbohydrate can be functionalized with amines which are subsequently reacted with bromoacetyl groups. The bromoacetylated carbohydrate is then reacted with thiolated protein. (Hermanson, G T, Bioconjugate Techniques, Academic Press, 2nd ed, 2008). The term “functionalization” generally means to chemically attach a group to add functionality, for example, to facilitate conjugation. Examples include functionalization of proteins with hydrazides or aminooxy groups and functionalization of carbohydrate with amino groups.

Embodiments of the invention can comprise two or more M-T7 polypeptides that are linked to each other (i.e., crosslinked). “Crosslinking” can refer to joining moieties together, such as M-T7 polypeptides, by either noncovalent or covalent bonds. For example, two or more polypeptides can be covalently linked by a linker. In embodiments, the linker can be a peptide linker. For example, the crosslinking comprises covalent crosslinking between polymers (i.e., polypeptides)

Embodiments of the invention also comprise nucleic acids encoding one or more M-T7 polypeptides. These polynucleotides include DNA, cDNA and RNA sequences which encode the peptide(s) of interest. Nucleic acid molecules encoding these peptides can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same peptide.

Nucleic acid sequences encoding one or more M-T7 polypeptide can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al, Meth. Enzymol. 68: 109-151, 1979; the diethylphosphoramidite method of Beaucage et al, Tetra. Lett. 22: 1859-1862, 1981 the solid phase phosphoramidite triester method described by Beaucage & Caruthers, Tetra. Letts. 22(20): 1859-1862, 1981, for example, using an automated synthesizer as described in, for example, Needham-VanDevanter et al, Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template.

Exemplary nucleic acids including sequences encoding one or more M-T7 polypeptides disclosed herein can be prepared by cloning techniques or chemical synthesis. Examples of appropriate cloning and sequencing techniques, and instructions sufficient to direct persons of skill through cloning are found in Sambrook et al, supra, Berger and Kimmel (eds.), supra, and Ausubel, supra. Product information from manufacturers of biological reagents and experimental equipment also provide useful information. Such manufacturers include the SIGMA Chemical Company (Saint Louis, MO), R&D Systems (Minneapolis, MN), Pharmacia Amersham (Piscataway, NJ), CLONTECH Laboratories, Inc. (Palo Alto, CA), Chem Genes Corp., Aldrich Chemical Company (Milwaukee, WI), Glen Research, Inc., GIBCO BRL Life Technologies, Inc. (Gaithersburg, MD), Fluka Chemica-Biochemika Analytika (Fluka Chemie AG, Buchs, Switzerland), Invitrogen (San Diego, CA), and Applied Biosystems (Foster City, CA), as well as many other commercial sources known to one of skill.

Once the nucleic acids encoding one or more M-T7 polypeptides are isolated and cloned, the peptide can be expressed in a recombinantly engineered cell such as bacteria, plant, yeast, insect and mammalian cells using a suitable expression vector or expressed in a viral vector for therapeutic approaches—eg Adeno-associated viral (AAV) vector expression.

One or more DNA sequences encoding one or more immunogenic peptide can be expressed in vitro by DNA transfer into a suitable host cell. The cell can be prokaryotic or eukaryotic. The term also includes any progeny of the subject host cell. In embodiments, the progeny are not identical to the parental cell since there can be mutations that occur during replication. Methods of stable transfer, meaning that the foreign DNA is continuously maintained in the host, are known in the art. In one example a vector is an adeno-associated virus (AAV) vectror.

The terms “recombinant nucleic acid” or “recombinantly produced nucleic acid” can refer to nucleic acids such as DNA or RNA which has been isolated from its native or endogenous source, and which can be modified, for example, chemically or enzymatically, by adding, deleting or altering naturally-occurring flanking or internal nucleotides. Flanking nucleotides are those nucleotides which are upstream or downstream from the described sequence or sub-sequence of nucleotides, while internal nucleotides are those nucleotides which occur within the described sequence or subsequence.

A “recombinant protein” is produced by “recombinant means”, which refers to techniques where proteins are isolated, the cDNA sequence coding the protein identified and inserted into an expression vector. The vector is then introduced into a cell and the cell expresses the protein. Recombinant means also encompasses the ligation of coding or promoter DNA from different sources into one vector for expression of a PPC, constitutive expression of a protein, or inducible expression of a protein.

Polynucleotide sequences encoding one or more M-T7 polypeptide can be operatively linked to expression control sequences (e.g., a promoter). An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The polynucleotide sequences encoding one or more M-T7 polypeptide can be inserted into an expression vector including, but not limited to a plasmid, virus or other vehicle that can be manipulated to allow insertion or incorporation of sequences and can be expressed in prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors that can express and replicate in a host are known in the art.

In an aspect, a composition disclosed herein comprises nucleic acid molecules that encode the M-T7-derived peptides or fragments thereof disclosed herein in an expression construct or in a single or separate cassette. Disclosed herein is an expression construct that can express M-T7-derived peptides or fragments thereof.

A disclosed expression cassette can include 5′ and 3′ regulatory sequences operably linked to a polynucleotide disclosed herein. “Operably linked” is intended to mean a functional linkage between two or more elements. For example, an operable linkage between a polynucleotide disclosed herein and a regulatory sequence (e.g., a promoter) is a functional link that allows for expression of a polynucleotide disclosed herein. Operably linked elements can be contiguous or non-contiguous. When used to refer to the joining of two protein coding regions, by operably linked is intended that the coding regions are in the same reading frame. An expression cassette can further comprise at least one additional polynucleotide to be co-transformed into the organism. Alternatively, one or more polypeptide(s) can be expressed on one or more expression cassettes. Expression cassettes can be provided with a plurality of restriction sites and/or recombination sites for insertion of the polynucleotide to be under the transcriptional regulation of the regulatory regions.

The regulatory regions (i.e., promoters, transcriptional regulatory regions, and translational termination regions) and/or the polynucleotides disclosed herein can be native/analogous to the host cell or to each other. Alternatively, the regulatory regions and/or the polynucleotide employed in the invention can be heterologous to the host cell or to each other. As used herein, “heterologous” in reference to a sequence can refer to a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

In preparing the expression cassette, the various DNA fragments can be manipulated, so as to provide for the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites, or the like. For this purpose, in vitro mutagenesis, primer repair, restriction, annealing, resubstitutions, e.g., transitions and transversions, can be involved,

A number of promoters can be used in the practice of the invention. The promoters can be selected based on the outcome. The choice of promoters depends on several factors including but not limited to efficiency, selectability, inducibility, expression level, and cell- or tissue-preferential expression. The nucleic acids can be combined with constitutive, tissue-preferred, inducible, or other promoters for expression in the host organism. One skilled in the art can appropriately select and position promoters and other regulatory regions relative to the coding sequence.

In addition to M-T7 polypeptide and/or nucleic acids encoding the M-T7 polypeptides, the topical formulation can further comprises one or more carriers and excipients, including viscosity increasing agents, ointment bases (e.g., cream bases), antimicrobial preservatives, temperature and pH sensing probes, emulsifying agents, and/or solvents.

A “viscosity increasing agent” can refer to an agent that is used to thicken a formulation. Exemplary viscosity increasing agents can include, for example, cetostearyl alcohol, cholesterol, stearyl alcohol, chlorocresol, white wax, stearic acid, cetyl alcohol, or a combination thereof. The viscosity increasing agent can be in the topical formation at a concentration of about 1.0-10% (w/w). For example, the topical formulation can comprise about 1-1.5%, 1.5-2%, 2-2.5%, 2.5-3%, 3-3.5%, 3.5-4%, 4-4.5%, 4.5-5%, 5-5.5%, 5.5-6%, 6-6.5%, 6.5-7%, 7-7.5%, 7.5-8%, 8-8.5%, 8.5-9%, 9-9.5%, or 9.5-10% (w/w) of the viscosity increasing agent. Alternatively, the topical formulation can comprise about 1-5%, 2.5-7.5%, or 5-10% (w/w) of the viscosity increasing agent.

An“ointment base” can be any semisolid preparation or vehicle into which an active agent can be incorporated. Exemplary ointment bases include, but are not limited to, oleaginous ointment bases (e.g., white petrolatum or white ointment), absorption ointment bases (e.g., hydrophilic petrolatum, anhydrous lanolin, Aquabase™, Aquaphor®, and Polysorb®), water/oil emulsion ointment bases (e.g., cold cream, hydrous lanolin, rose water ointment, Hydrocream™, Eucerin®, and Nivea®), oil/water emulsion ointment bases (e.g., hydrophilic ointments, Dermabase™, Velvachol®, and Unibase®), and water- miscible ointment bases (e.g., polyethylene glycol (PEG) ointment and Polybase™). Ointment bases can be pharmacologically inert but can entrap water in order to provide an emollient protective film. In an embodiment, the ointment base can be any petrolatum compound (e.g., petrolatum, white petrolatum, white soft paraffin, liquid petrolatum, liquid paraffin). In a further specific embodiment, the ointment base is white petrolatum (CAS number 8009-03-8). The ointment base can be in the topical formation at a concentration of about 5-30% (w/w), e.g., 10-30% (w/w). For example, the topical formulation can comprise about 5-25%, 5-20%, 5-15%, 5-15%, 10-15%, 15-20%, 20-25%, or 25-30% (w/w) of the ointment base. For example, the topical formulation can comprise about 5, 6, 7, 8, 9, 10, 11, 12, 13, 14, 15, 16, 17, 18, 19, 20, 21, 22, 23, 24, 25, 26, 27, 28, 29, or 30 percent (w/w) of the ointment base.

In some embodiments, the“ointment base” described herein contains less than 20% water and volatiles, and more than 50% hydrocarbons, waxes, or polyols as the vehicle.

In some embodiments, the“ointment base” described herein is a “cream base,” which contains more than 20% water and volatiles and/or can contain less than 50% hydrocarbons, waxes, or polyols as the vehicle for the drug substance. The cream base can be a multiphase preparation containing a lipophilic phase and an aqueous phase. In some instances, the cream base is a lipophilic cream base, which has a lipophilic phase as the continuous phase. Such a cream base can contain water-in-oil emulsifying agents such as wool alcohols, sorbitan esters and monoglycerides. In other instances, the cream base is a hydrophilic cream base, which has an aqueous phase as the continuous phase. Such a cream base can contain oil-in-water emulsifying agents such as sodium or trolamine soaps, sulfated fatty alcohols, polysorbates and polyoxyl fatty acid and fatty alcohol esters, which can be in combination with water-in-oil emulsifying agents, if needed.

As used herein, the term “aqueous solution” can refer to a solution, wherein at least one solvent is water and the weight % of water in the mixture of solvents is at least 50%, at least 60%, at least 70% or at least 90%. In some embodiments, aqueous solution is a solution in which water is the only solvent. In some embodiments, aqueous solution is a buffer (e.g., phosphate buffer or a carbonate buffer). In some embodiments, the buffer is physiological buffer or a pharmaceutically acceptable buffer. In some embodiments, the buffer is any one of buffers described, for example, in Y.-C. Lee et al. International Journal of Pharmaceutics 253 (2003) 111-119, the disclosure of which is incorporated herein by reference in its entirety. In some embodiments, the buffer comprises maleic acid, tartaric acid, lactic acid, citric acid, acetic acid, sodium bicarbonate, sodium phosphate, or mixtures thereof. In some embodiments, the pH range of the buffer is from about 3 to about 9, from about 4 to about 8, from about 5 to about 7, from about 6 to about 7, from about 3 to about 5, from about 3 to about 7, from about 4 to about 6, or from about 6 to about 6. In some embodiments, the pH of the buffer is about 4, about 5, about 6, about 6.4, about 6.5, about 6.6, about 7, about 7.5, or about 8.

An “antimicrobial preservative” can be any compound that can destroymicrobes, prevent the multiplication or growth of microbes, or prevent the pathogenic action of microbes. Exemplary antimicrobial preservatives include, but are not limited to, a paraben compound (an ester of para-hydroxybenzoic acid; e.g., paraben, methylparaben, ethylparaben, propylparaben, butylparaben, heptylparaben, benzylparaben, isobutylparaben, isopropylparaben, benzylparaben, or their sodium salts), benzalkonium chloride, benzethonium chloride, benzyl alcohol, boric acid, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. The antimicrobial preservative can be present in the topical formation at a concentration of about 0.005-0.2%, e.g., about 0.01-0.2% (w/w). For example, the topical formulation can comprise about 0.005-0.01%, 0.01-0.05%, 0.05-0.1%, 0.15%, or 0.15-0.2% (w/w) of the antimicrobial preservative. For example, the topical formulation can comprise about 0.005, 0.006, 0.007, 0.008, 0.009, 0.01, 0.02, 0.03, 0.04, 0.06, 0.07, 0.08, 0.09, 0.1, 0.11, 0.12, 0.13, 0.14, 0.15, 0.16, 0.17, 0.18, 0.19, or 0.2 percent (w/w) of the antimicrobial preservative.

An“emulsifying agent” can refer to a compound or substance which acts as a stabilizer for a mixture of two or more liquids that are normally immiscible (unmixable or unblendable). Exemplary emulsifying agents can include, but are not limited to, natural emulsifying agents (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, propylene glycol monostearate, and polyvinyl alcohol), carbomers (e.g., carboxy polymethylene, polyacrybc acid, acrylic acid polymer, and carboxy vinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, and methylcellulose), sorbitan fatty acid esters (e.g., polyoxyethylene sorbitan monolaurate [Tween® 20], polyoxyethylene sorbitan [Tween® 60], polyoxyethylene sorbitan monooleate [Tween® 80], sorbitan monopalmitate [Span® 40], sorbitan monostearate [Span® 60], sorbitan tristearate [Span® 65], glyceryl monooleate, and sorbitan monooleate [Span® 80]), polyoxyethylene esters (e.g., polyoxyethylene monostearate [Myq® 45], polyoxyethylene hydrogenated castor oil, polyethoxylated castor oil, polyoxymethylene stearate, and Solutol), sucrose fatty acid esters, polyethylene glycol fatty acid esters (e.g., Cremophor®), polyoxyethylene ethers (e.g., polyoxyethylene lauryl ether [Brij® 30]), and poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, and docusate sodium, and/or combinations thereof. The emulsifying agent can be present in the topical formation at a concentration of about 0.5-10% (w/w), e.g., (w/w). For example, the topical formulation can comprise about 0.5-1%, 1-1.5%, 1.5-2%, 2-2.5%, 2.5-3%, 3-3.5%, 3.5-4%, 4-4.5%, 4.5-5%, 5- 5.5%, 5.5-6%, 5-10%, 6-10%, or 8-10% (w/w) of the emulsifying agent. For example, the topical formulation can comprise about 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or percent (w/w) of the emulsifying agent.

The topical formulation of the invention can further contain one or more solvents (e.g., non-water solvents or water). Exemplary non-water solvents can include, but are not limited to, any known solvent including propylene glycol, glycol, and mixtures thereof. The non-water solvent can be present in the topical formation at a concentration of about 2-65% (w/w). For example, the topical formulation can comprise about 2-15%, 15-30%, 30-45%, or (w/w) of the solvent. In some embodiments, the topical formulation of the invention can also contain water.

In some embodiments, the topical formulation of the invention can further comprise one or more emollients, fragrances, or pigments. The topical formula can also be used in conjunction with a wound dressing (e.g., bandage with adhesive, plaster patch and the like) (e.g., cyclohexane, n-hexane, n-decane, i-octane, octane, butyl ether, carbon tetrachloride, triethyl amine, i-propyl ether, toluene, p-xylene, t-butyl methyl ether, benzene, benzyl ether, dichloromethane, methylene chloride, chloroform, dichloroethane, ethylene di chloride, 1 -butanol, i-butyl alcohol, tetrahydrofuran, ethyl acetate, 1 -propanol, 2-propanol, methyl acetate, cyclohexanone, methyl ethyl ketone (MEK), nitrobenzene, benzonitrile, 1,4-dioxane, or p-dioxane). In certain embodiments, the topical formulation includes a hydrogel.

In embodiments, the topical formulation further comprises one or more additional active ingredients. For example, the one or more additional active ingredients comprises an antibiotic, a pain reliever, an anti-inflammatory, an anti-scarring agent, a moisturizer, a steroid, an immune modulator, or a growth factor. Non-limiting examples of the active ingredient comprise human serum albumen, calcium, bovine thrombin, human Thrombin (hThrombin), rhThrombin, factor Vila, factor XIII, recombinant Factor XIII (rF actor XIII), thromboxane A2, prostaglandin-2a, epidermal growth factor, platelet derived growth factor, Von Willebrand factor, tumor necrosis factor (TNF), TNF-alpha, transforming growth factor (TGF), TGF-alpha, TGF-beta, insulin like growth factor, fibroblast growth factor, keratinocyte growth factor, nerve growth factor,

In embodiments, a topical formulation can include an antibiotic, including antimicrobial peptides (AMP). “Antibiotic” can refer to a substance that controls the growth of bacteria, fungi, or similar microorganisms, wherein the substance can be a natural substance produced by bacteria or fungi, or a chemically/biochemically synthesized substance (which may be an analog of a natural substance), or a chemically modified form of a natural substance. In general, any antibiotic can be used with the disclosed composition or methods. Examples of antibiotics that can be used include but are not limited to aminoglycosides (such as amikacin, gentamicin, kanamycin, neomycin, netilmicin, streptomycin, tobramycin, and paromomycin); ansamycins (such as geldanamycin, and herbimycin); carbacephems (such as loracarbef, ertapenem, doripenem, imipenem/cilastatin, and meropenem); cephalosporins (such as cefadroxil, cefazobn, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, and ceftobiprole); gly copeptides (such as teicoplanin and vancomycin); macrolides (such as azithromycin, clarithromycin, dirithromycin, erythromycin, roxithromycin, troleandomycin, telithromycin, and spectinomycin); monobactams (such as aztreonam); penicillins (such as amoxicillin, ampicillin, azlocillin, carbenicillin, cloxacillin, dicloxacillin, flucloxacillin, mezlocillin, meticillin, amoxycillin, clavamox, clavulanic acid, nafcillin, oxacillin, penicillin, piperacillin, and ticarcillin); peptides (such as bacitracin, colistin, and polymyxin b); quinolones (such as ciprofloxacin, enoxacin, gatifloxacin, levofloxacin, lomefloxacin, moxifloxacin, norfloxacin, ofloxacin, trovafloxacin, grepafloxacin, and sparfloxacin); sulfonamides (such as mafenide, prontosil (archaic), sulfacetamide, sulfamethizole, sulfanilimide (archaic), sulfasalazine, sulftsoxazole, trimethoprim, and trimethoprim-sulfamethoxazole); tetracyclines (such as demeclocycline, doxycycline, minocycline, oxy tetracycline, and tetracycline); and others (such as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampicin, thiamphenicol, and tinidazole) or combinations thereof

Other active ingredients which can be included in a topical formulation, such as that described herein, include a pain reliever, an anti-inflammatory, an anti-scarring agent, a moisturizer, a steroid, an immune modulator, or a growth factor.

For example, the term “pain reliever” or “pain relieving agent” can refer to one having an action of relieving pain. Non-limiting examples of pain relievers, can include acetaminophen, ibuprofen, ketoprofen, diclofenac, naproxen, aspirin, and combinations thereof, as well as prescription analgesics, non-limiting examples of which include propyxhene HCl, codeine, mepridine, and combinations thereof.

For example, “immune modulator” can refer to a substance that can alter (e.g., inhibit, decrease, increase, enhance or stimulate) the working of any component of the innate, humoral or cellular immune system of a mammal. For example, the “immune modulator” can be SERP-1, or other immune modulators derived from natural sources.

For example, the term “growth factor” can refer to proteins that promote growth, and include, for example, hepatic growth factor; fibroblast growth factor; vascular endothelial growth factor; nerve growth factors such as NGF-β; platelet-derived growth factor; transforming growth factors (TGFs) such as TGF-a and TGF-β; insulin-like growth factor-I and -II; erythropoietin (EPO); osteoinductive factors; interferons such as interferon-α, -β, and -y; and colony stimulating factors (CSFs) such as macrophage-CSF (M-CSF), granulocyte-macrophage-CSF (GM-CSF), and granulocyte-CSF (G-CSF). As used herein, the term growth factor includes proteins from natural sources or from recombinant cell culture and biologically active equivalents of the native-sequence growth factor, including synthetically produced small-molecule entities and pharmaceutically acceptable derivatives and salts thereof.

Similar to the most therapeutic proteins, M-T7 polypeptide can exhibit a short half-live and low stability. Thus, it can be desirable to control M-T7 protein release in order to potentially extend the release time (i.e., delayed release) and increase stability for a long-term topical treatment, such as wound healing. To combat this potential problem, embodiments described herein comprise M-T7 polypeptides with hydrogels to form slow release composition. Thus, embodiments are also are directed to hydrogels that include a M-T7 polypeptide, or nucleic acid encoding a M-T7 polypeptide, and optionally, other active ingredients as discussed herein. In some examples the hydrogels are incorporated into a wound dressing for promoting wound healing. To this end, aspects of the disclosure are directed to wound dressings, and methods of using such wound dressings.

A hydrogel is a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include polysaccharides such as chitosan, alginate, polyphosphazenes, and polyacrylates such as hydroxyethyl methacrylate, which are crosslinked ionically, or block copolymers such as PLURONICS™ (BASF Corporation) or TETRONICS™ (BASF Corporation), polyethylene oxide-polypropylene glycol block copolymers which are crosslinked by temperature or pH, respectively. Other materials include proteins such as fibrin, polymers such as polyvinylpyrrolidone, hyaluronic acid and collagen.

The hydrogel can also include gelatin, cellulose, or collagen-based materials. In some examples, the gelatin-based substrate includes an absorbable sponge, powder or film of cross-linked gelatin, for example, GELFOAM® (Upjohn, Inc., Kalamazoo, Mich.) which is formed from denatured collagen. A cellulose-based substrate includes an appropriate absorbable cellulose such as regenerated oxidized cellulose sheet material, for example, SURGICEL® (Johnson & Johnson, New Brunswick, N.J.) or Oxycel® (Becton Dickinson, Franklin Lakes, N.J.). Collagen materials can include an appropriate resorbable collagen, such as purified bovine corium collagen, for example, AVITENE® (MedChem, Wobum, Mass.), HELISTAT® (Marion Merrell Dow, Kansas City, Mo.), HEMOTENE® (Astra, Westborough, Mass.), or SURGIFOAM® (Johnson & Johnson, New Brunswick, NJ). There have been prior success with the application of a chitosan bandage (see for example, HemCon®, Tricol Biomedical Inc.) for wound healing.

Chitosan-based hydrogels, such as chitosan-collagen hydrogel, have also been tested for wound treatment for delivery of antimicrobials, peptides, and growth factors showing significant promotion on wound healing (Liu et al, RSC Adv. (2018), Elviri et al, Expert Opin. Drug Deliv. (2017), Hamedi et al, Carbohydr. Polym. (2018), Riva et al., Adv. Polym. Sci. (2011), Liu et al, Adv. Polym. Sci. (2011)). Considering the biocompatible, antimicrobial, biologically adhesive, hemostatic effect and applications for drug delivery, a chitosan-based hydrogel as a drug delivery system for the treatment of wound healing with M-T7 is disclosed herein. In addition to the discovery of function of M-T7 in promoting wound healing as described herein, a chitosan-collagen hydrogel carrier can efficiently deliver M-T7 locally to a wound site and promote healing. Thus, in some embodiments, an M-T7 polypeptide, or nucleic acid encoding an M-T7 polypeptide (and other active ingredients as described herein) are incorporated into a chitosan-collagen hydrogel carrier.

During wound healing, collagen accumulation and organization are correlated with scar formation. Collagens play a crucial role in angiogenesis during tissue regeneration. It is well known that collagen I is a central factor allowing for endothelial cells to initiate precapillary cord formation. In contrast increased deposition of collagen III reduces the density of blood vessels at sites of wound healing (Davis and Senger et al., Circ. Res. (2005), O'Rourke et al, Adv. Wound Care. (2018)).

In embodiments, the wound dressing that includes a M-T7 polypeptide or nucleic acid encoding a M-T7 polypeptide is formed of a biomaterial, such as poly [b-(1-4)-2-amino-2-deoxy-D-glucopyranose], which can be referred to as chitosan, and, in embodiments, in combination with collagen, e.g. collagen -chitosan hydrogels. The wound dressing can be formed into a sponge-like or woven configuration via the use of an intermediate structure or form producing steps. The biomaterial comprises an interconnected open porous structure, and/or an oriented open lamella structure, and/or an open tubular structure, and/or an open honeycomb structure, and/or a filamentous structure.

Embodiments can include those that comprise a sustained release or controlled release matrix. In addition, embodiments can be used in conjunction with other treatments that use sustained-release formulations. As used herein, a sustained-release matrix can refer to a matrix made of materials, usually polymers, which are degradable by enzymatic or acid-based hydrolysis or by dissolution. Once inserted into the body, the matrix is acted upon by enzymes and body fluids. A sustained-release matrix can be chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system.

In another embodiment, the compositions of the present disclosure (as well as combination compositions separately or together) include those formed by impregnation of the composition or pharmaceutical composition described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

Aspects of the invention are further directed towards a bandage, wound dressing, or graft permeated with an M-T7 polypeptide for in vivo use. As used herein, “in vivo use” can refer to a use wherein the M-T7 polypeptide permeated graft is at least partially positioned on or within the body of a subject. For example, use of an M-T7 polypeptide permeated graft placed on a wound of a subject to facilitate wound healing can be considered an in vivo use. Similarly, use of an M-T7 polypeptide permeated graft implanted within a subject following a surgical procedure to facilitate tissue regeneration can be considered an in vivo use.

The bandage, wound dressing, or graft can comprise a bioscaffold permeated with M-T7 polypeptide. As used herein, “bioscaffold” can refer to a substrate on which cells can grow. In embodiments, the bioscaffold can mimic the native biological extracellular matrix of the tissue it is meant to regenerate.

In an embodiment, the bandage, wound dressing, or graft can comprise a hydrogel. A hydrogel is a three-dimensional solid that comprises a network of hydrophilic polymer chains that results from the hydrophilic polymer chains being held together by cross-links. Because of the inherent cross-links, the structural integrity of the hydrogel network does not dissolve from the high concentration of water. Hydrogels are highly absorbent (they can contain over 90% water) natural or synthetic polymeric networks. Hydrogels also possess a degree of flexibility very similar to natural tissue, due to their significant water content. Biohydrogels are known in the art, and have been developed for a broad scope of therapeutic applications, such as for the release of Biomacromolecules or drugs, wound healing, or as a barrier for contact lenses or ocular surface injuries. See, for example, Mateescu, Mihaela, et al. “Antibacterial peptide-based gel for prevention of medical implanted-device infection.” PLoS One10.12 (2015): e0145143; Zhao, Fan, Man Lung Ma, and Bing Xu. “Molecular hydrogels of therapeutic agents.” Chemical Society Reviews 38.4 (2009): 883-891; each of which are incorporated herein by reference in their entireties.

In embodiments, the bandage, wound dressing, or graft can comprise a “biodegradable polymer”, which can refer to a polymer which may be broken down into organic substances, such as by living organisms. For example, the bandage, wound dressing, or graft can comprise biodegradable polymers such as chitosan, collagen, fibrin, polyarginine, polylysine, alginate, cyanoacrylate, dermabond, and the like, and combinations thereof. For example, a bandage, wound dressing, or graft can partially or completely comprises one or more biodegradable polymers.

In embodiments, the polymer can be a natural polymer or a synthetic polymer. Natural polymers occur in nature and can be extracted, such as polysaccharides or proteins. Non-limiting examples of polysaccharides comprise chondroitin sulfate, heparin, heparan, alginic acid (i.e., alginate), hyaluronic acid, dermatan, dermatan sulfate, pectin, carboxymethyl cellulose, chitosan, melanin (and its derivatives, such as eumelanin, pheomelanin, and neuromelanin), agar, agarose, gellan, gum, and the like as well as their salt forms (such as sodium salt and potassium salt). Non-limiting examples of proteins comprise collagen, alkaline gelatin, acidic gelatin, gene recombination gelatin, and so on.

Synthetic polymers are man-made molecules formed by the polymerization of a variety of monomers, such as macromolecules comprising polyacrylic acid, polyaspartic acid, polytartaric acid, polyglutamic acid, polyfumaric acid, polyarginine, polylysine, polyhistidine, and so on as well as their salt forms (such as sodium salt and potassium salt). Non-limiting examples of synthetic polymers comprise cyanoacrylate, pluronic diacrylate, amino acid-based poly(ester amide) polymers (such as those based on arginine, lysine, or histidine).

In embodiments, the polymer can be a cationic polymer, which can refer to a polymer with a positive charge. For example, the graft comprises cationic polymers such as polyarginine, polylysine, or polyhistidine, and the like. The skilled artisan will recognize that the polymer can be any polymer that is suitable to form electrostatic nanocomplexes with negatively charged compounds or neutral compounds.

Methods for Promoting Wound Healing

Any of the formulations (e.g., topical formulations) described herein can be used for treating and/or promoting wound healing in a subject in need of the treatment. As used herein, the terms “treat,” “treating” or “treatment” can refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, delay of the onset of the disease, disorder, or illness, and/or change in clinical parameters of the condition, disorder, disease or illness, etc., as can be well known in the art.

The term “wound” can refer to an injury to living tissue caused by a cut, blow, or other impact (e.g., caused by a medical condition such as a skin disorder), such as one in which the skin is cut or broken. Any disruption of normal anatomy, from whatever cause, can be considered a wound. Causes of wounds can include but are not limited to traumatic injuries such as mechanical, thermal, and incisional injuries; elective injuries such as surgery and resultant incisional hernias; acute wounds, chronic wounds, infected wounds, dermal wounds, and sterile wounds, as well as wounds associated with disease states (i.e. ulcers caused by diabetic neuropathy; skin disorders).

Wounds contemplated by the invention include cuts and lacerations, surgical incisions or wounds, punctures, grazes, scratches, compression wounds, abrasions, friction wounds (e.g. nappy rash, friction blisters), decubitus ulcers (e.g. pressure or bed sores); thermal effect wounds (burns from cold and heat sources, either directly or through conduction, convection, or radiation, and electrical sources), chemical wounds (e.g. acid or alkali burns) or pathogenic infections (e.g. viral, bacterial or fungal) including open or intact boils, skin eruptions, blemishes and acne, ulcers, chronic wounds, (including diabetic-associated wounds such as lower leg and foot ulcers, venous leg ulcers and pressure sores), skin graft/transplant donor and recipient sites, immune response conditions, e.g. psoriasis and eczema, stomach or intestinal ulcers, oral wounds, including a ulcers of the mouth, damaged cartilage or bone, amputation wounds and corneal lesions.

In embodiments, the wound is a transplant wound. In embodiments, the wound is not a transplant wound.

For example, the wound can comprise a burn wound, a surgical wound, a diabetic ulcer, a pressure ulcer, an ischemic wound, a venous and/or arterial ulcer, or a chronic wound.

A wound is dynamic and the process of healing is a continuum requiring a series of integrated and interrelated cellular processes that begin at the time of wounding and proceed beyond initial wound closure through arrival at a stable scar. These cellular processes are mediated or modulated by humoral substances including but not limited to cytokines, lymphokines, growth factors, and hormones.

The term “wound healing” can refer to the dynamic and complex process of replacing devitalized or missing cellular structures and/or tissue layers. In embodiments, wound healing can refer to improving, by some form of intervention, the natural cellular processes and humoral substances such that healing is faster, and/or the resulting healed area has less scaring and/or the wounded area possesses tissue tensile strength that is closer to that of uninjured tissue.

The term “promotion of wound healing” or “promoting wound healing” can refer to the inducement of an increased level or rate of replacement for devitalized or missing cellular structures and/or tissue layers. As an example, promotion of wound healing can be indicated by partial or complete ulcer closure or an increase in the healing rate of an ulcer (including but not limited to more rapid changes in ulcer size, area, or severity, a more rapid closure of the ulcer, and/or an increase in the percentage change from baseline in ulcer size, area, or severity when compared to a control ulcer treated with a placebo).

As used herein, the term “dermal wound” can refer to an injury to the skin in which the skin is cut or broken.

In embodiments, the wound can be any internal wound, e.g. where the external structural integrity of the skin is maintained, such as in bruising or internal ulceration, or external wounds, particularly cutaneous wounds, and consequently the tissue may be any internal or external bodily tissue. In one embodiment the tissue is skin (such as human skin), i.e. the wound is a cutaneous wound, such as a dermal or epidermal wound.

The human skin is composed of two distinct layers, the epidermis and the dermis, below which lies the subcutaneous tissue. The primary functions of the skin are to provide protection to the internal organs and tissues from external trauma and pathogenic infection, sensation and thermoregulation.

The outermost layer of skin, the epidermis, is approximately 0.04 mm thick, is avascular, is comprised of four cell types (keratinocytes, melanocytes, Langerhans cells, and Merkel cells), and is stratified into several epithelial cell layers. The inner-most epithelial layer of the epidermis is the basement membrane, which is in direct contact with, and anchors the epidermis to, the dermis. All epithelial cell division occurring in skin takes place at the basement membrane. After cell division, the epithelial cells migrate towards the outer surface of the epidermis. During this migration, the cells undergo a process known as keratinization, whereby nuclei are lost and the cells are transformed into tough, flat, resistant non-living cells. Migration is completed when the cells reach the outermost epidermal structure, the stratum corneum, a dry, waterproof squamous cell layer which helps to prevent desiccation of the underlying tissue. This layer of dead epithelial cells is continuously being sloughed off and replaced by keratinized cells moving to the surface from the basement membrane. Because the epidermal epithelium is avascular, the basement membrane is dependent upon the dermis for its nutrient supply.

The dermis is a highly vascularized tissue layer supplying nutrients to the epidermis. In addition, the dermis contains nerve endings, lymphatics, collagen protein, and connective tissue. The dermis is approximately 0.5 mm thick and is composed predominantly of fibroblasts and macrophages. These cell types are largely responsible for the production and maintenance of collagen, the protein found in all animal connective tissue, including the skin. Collagen is primarily responsible for the skin's resilient, elastic nature. The subcutaneous tissue, found beneath the collagen-rich dermis, provides for skin mobility, insulation, calorie storage, and blood to the tissues above it.

Wounds can be classified in one of two general categories, partial thickness wounds or full thickness wounds. A partial thickness wound is limited to the epidermis and superficial dermis with no damage to the dermal blood vessels. A full thickness wound involves disruption of the dermis and extends to deeper tissue layers, involving disruption of the dermal blood vessels. The healing of the partial thickness wound occurs by simple regeneration of epithelial tissue. Wound healing in full thickness wounds is more complex. Cutaneous wounds contemplated by the invention may be either partial thickness or full thickness wounds.

The term “chronic wound” can refer to a wound that has not healed. For example, a wound that does not heal within 1 month, 2 months, 3 months, or longer than 3 months is considered chronic. Chronic wounds, including pressure sores, venous leg ulcers and diabetic foot ulcers, can simply be described as wounds that fail to heal. Whilst the exact molecular pathogenesis of chronic wounds is not fully understood, it is acknowledged to be multi-factorial. As the normal responses of resident and migratory cells during acute injury become impaired, these wounds are characterized by a prolonged inflammatory response, defective wound extracellular matrix (ECM) remodeling and a failure of re-epithelialization.

An “infected wound” can refer to a wound in which bacteria and/or other microorganisms are grown and infiltrated in the wound part. Infected wounds are conditions that have obvious signs of inflammation and delay healing.

A “burn wound” can refer to a case where a large surface area of an individual's skin has been removed or lost due to heat and/or chemical agents.

Diabetes can cause wound to heal more slowly, thereby increasing the risk that people with diabetes will develops infections. The term “diabetic wound” refers to any wound in an individual having diabetes, including chronic wounds occurring in diabetic patients.

The topical formulation can be applied to a wound site following a suitable dosage and treatment regimen. The dosage and administration regimen for the described method will depend on the nature and condition of the wound being treated, the age and condition of the patient, and any prior or concurrent therapy. In some instances, the topical formulation can be applied once every week, once every other day, once daily, twice daily, three times daily, or four time daily for a suitable period of time. The treatment can be terminated when the wound is recovered. When necessary, the treatment can resume, for example, if a wound recurs.

The subject to be treated by the topical formulation can be a human or a non-human mammal. As used herein, the term “subject” and “patient” are used interchangeably herein and can refer to both human and nonhuman animals. The term “nonhuman animals” of the disclosure includes vertebrates, e.g., mammals and non-mammals, such as nonhuman primates, sheep, dog, cat, horse, cow, rodents (e.g., mice, rats, etc.) and the like. For example, the subject is a human patient. In embodiments, the subject of this disclosure is a human subject. A “subject in need thereof” or “a subject in need of” is a subject known to have, or is suspected of having a surface wound, such as a wound in the skin and surrounding tissue.

In some embodiments, the subject is a human patient having an open wound, which can refer to an injury or damage to living tissues (e.g., skin) that cause a disruption in the normal continuity of biological structures. An open wound can include, but is not limited to, an abrasion, incision, laceration, puncture, avulsion, cut, or other similar injuries.

In other embodiments, the subject is a human patient having a chronic wound, which can be injuries or damage to living tissues (e.g., skin) that cause a disruption in the normal continuity of biological structures and do not heal in an orderly set of stages and/or in a predictable amount of time. A chronic wound can include, but is not limited to: a surgical wound, a traumatic wound, a pressure ulcer, a venous ulcer, or a diabetic ulcer. In other examples, a chronic wound can be associated with a disease or disorder, for example, a carcinoma, bum, bedsore, a skin disorder such as atopic dermatitis.

In one example, the subject is a human patient having an ulcer, such as a foot ulcer, associated with diabetes (e.g., type I or type II). Diabetes mellitus (also known as diabetes) is a group of metabolic diseases which result in high blood sugar levels over a prolonged period. Diabetes can result from the pancreas not producing enough insulin or the cells of the body not responding properly to the insulin produced. The three main types of diabetes mellitus are Type I (also known as “insulin-dependent diabetes mellitus” (IDDM) or“juvenile diabetes”; results from the failure of the pancreas to produce enough insulin), Type 2 (also known as“non-insulin-dependent diabetes mellitus” (NIDDM) or“adult-onset diabetes”; results from the failure of cells to respond to insulin properly), and gestational diabetes (seen during pregnancy when high blood sugar levels are observed in the absence of a previous history of diabetes). Many serious complications are observed in diabetic patients including, but not limited to, chronic wounds such as diabetic foot ulcers (also known as diabetic ulcers).

In some embodiments, the subject to be treated by the methods described herein suffers from a severe wound, for example, having an ulcer with an area greater than 2 cm² (e.g., 3 cm², 4 cm² or 5 cm²). In some examples, the subject suffers from one or more plantar ulcers.

Embodiments as described herein can be administered to a subject in one or more doses. Those of skill will readily appreciate that dose levels can vary as a function of the specific the formulation or pharmaceutical composition administered, the severity of the wound, the severity of the symptoms and the susceptibility of the subject to side effects. Advantageous dosages for a given compound are readily determinable by those of skill in the art by a variety of means.

In an embodiment, multiple doses of the formulation or pharmaceutical composition can be administered. The frequency of administration of the formulation or pharmaceutical composition can vary depending on any of a variety of factors, e.g., the wound, the severity of symptoms, and the like. For example, in an embodiment, the formulation or pharmaceutical composition can be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), or four times a day. In an embodiment, the formulation or pharmaceutical composition is administered 1 to 4 times a day over a 1 to 10-day time period.

The duration of administration of the formulation or pharmaceutical composition, e.g., the period of time over which the formulation or pharmaceutical composition is administered, can vary, depending on any of a variety of factors, e.g., patient response, etc. For example, the formulation or pharmaceutical composition in combination or separately, can be administered over a period of time of about one day to one week, about one day to two weeks.

The amount of the formulations and pharmaceutical compositions of the disclosure that can be effective in treating the condition or disease can be determined by standard clinical techniques. In addition, in vitro or in vivo assays can be employed to help identify optimal dosage ranges. The precise dose to be employed can also depend on the route of administration, and can be decided according to the judgment of the practitioner and each patient's circumstances.

Embodiments of the disclosure provide methods and compositions for the administration of the active agent(s) to a subject (e.g., a human) using any available method and route suitable for drug delivery, including in vivo and ex vivo methods, as well as systemic and localized routes of administration. Routes of administration include intranasal, intramuscular, intratracheal, subcutaneous, intradermal, intravitreal, topical application, intravenous, rectal, nasal, oral, and other enteral and parenteral routes of administration. Routes of administration can be combined, if desired, or adjusted depending upon the agent and/or the desired effect. An active agent can be administered in a single dose or in multiple doses.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to affect systemic or local delivery of the composition. Where systemic delivery is desired, administration typically involves invasive or systemically absorbed topical or mucosal administration of pharmaceutical preparations. In an embodiment, the composition or pharmaceutical composition can also be delivered to the subject by enteral administration. Enteral routes of administration include, but are not limited to, oral and rectal (e.g., using a suppository) delivery.

Methods of administration of the formulation or pharmaceutical composition through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

In embodiments, the M-T7 polypeptide permeated bandage or wound dressing can be implanting onto a prepared site on or within a subject in need thereof; thereby grafting to a subject the polymer-permeated graft. In other embodiments, the M-T7 polypeptide permeated bandage or wound dressing can be implanted onto a site on or within a subject prior to such site being cleaned and/or prepared, such as in an emergency setting. In such instances, the M-T7 polypeptide permeated bandage or wound dressing can prevent subsequent infect, reducing scarring, and/or prepare the site for healing.

Kits for Use in Promoting Wound Healing

The disclosure also provides kits for use in promoting wound healing. Such kits can include one or more containers comprising a topical formulation as described herein, which comprises a disclosed M-T7 polypeptide and/or a nucleic acid molecule encoding a M-T7 polypeptide.

In some embodiments, the kit can comprise instructions for use in accordance with any of the methods described herein. The included instructions can comprise a description of administration of the topical formulation to promote wound healing according to any of the methods described herein. The kit can further comprise a description of selecting an individual suitable for treatment based on identifying whether that individual has wounds in need of treatment.

The instructions relating to the use of a topical formulation can include information as to dosage, dosing schedule, and route of administration for the intended treatment. The containers can be unit doses, bulk packages (e.g., multi-dose packages) or sub-unit doses. Instructions supplied in the kits of the invention are can be written instructions on a label or package insert (e.g., a paper sheet included in the kit), but machine-readable instructions (e.g., instructions carried on a magnetic or optical storage disk) are also acceptable.

The label or package insert indicates that the composition is used for promoting wound healing. Instructions can be provided for practicing any of the methods described herein.

The kits of this invention are in suitable packaging. Suitable packaging includes, but is not limited to, vials, bottles, jars, flexible packaging (e.g., sealed Mylar or plastic bags), and the like. At least one active agent in the composition is an active agent selected from the group consisting of a M-T7 polypeptide and/or a nucleic acid molecule encoding a disclosed M-T7 polypeptide.

Kits can optionally provide additional components such as interpretive information. Normally, the kit comprises a container and a label or package insert(s) on or associated with the container. In some embodiments, the invention provides articles of manufacture comprising contents of the kits described herein.

### Other Embodiments

Other compositions, compounds, methods, features, and advantages of the disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Recombinant Myxoma Virus-Derived Immune Modulator M-T7 Accelerates Cutaneous Wound Healing and Improves Tissue Remodeling

Complex dermal wounds represent major medical and financial burdens, especially in the context of comorbidities such as diabetes, infection and advanced age. New approaches to accelerate and improve, or “fine tune” the healing process, so as to improve the quality of cutaneous wound healing and management, are the focus of intense investigation. Here, we investigate the topical application of a recombinant immune modulating protein which inhibits the interactions of chemokines with glycosaminoglycans, reducing damaging or excess inflammation responses in a splinted full-thickness excisional wound model in mice. M-T7 is a 37 kDa-secreted, virus-derived glycoprotein that has demonstrated therapeutic efficacy in numerous animal models of inflammatory immunopathology. Topical treatment with recombinant M-T7 significantly accelerated wound healing when compared to saline treatment alone. Healed wounds exhibited properties of improved tissue remodeling, as determined by collagen maturation. M-T7 treatment accelerated the rate of peri-wound angiogenesis in the healing wounds with increased levels of TNF, VEGF and CD31. The immune cell response after M-T7 treatment was associated with a retention of CCL2 levels, and increased abundances of arginase-1-expressing M2 macrophages and CD4 T cells. Thus, topical treatment with recombinant M-T7 promotes a pro-resolution environment in healing wounds, and, without wishing to be bound by theory, can be a new treatment approach for cutaneous tissue repair.

Introduction

Dermal wounds, especially those complicated by factors such as diabetes, infection and age, represent a major medical burden, estimated to account for an annual expenditure of more than USD 20 billion by 2024 [1]. New approaches to manage and improve the healing of cutaneous wounds are the focus of intense investigation. As the first barrier protecting the host from external insults, the skin contains an intricate immune system that rapidly responds to limit infection, to remove debris and to repair damage. Cutaneous wound healing is a complex multi-stage process that relies upon numerous cell types and mediators acting in a coordinated temporal sequence. The wound healing process can be described in four phases: (i) hemostasis (ii) inflammation, (iii) tissue generation (proliferation) and (iv) remodeling [2]. Dysregulation at any stage of wound healing prolongs the resolution process, worsens scarring and leads to tissue disruption and the risk of infection. The immune system plays a critical role in successful wound healing. In addition to contributing to host defenses against infection, immune cells are critical regulators of wound healing through the secretion of cytokines, chemokines and growth factors that orchestrate local inflammatory cell invasion and responses, cellular differentiation, and tissue regeneration [3,4]. The immune cells involved in wound healing include classes of neutrophils, macrophages, and T and B lymphocytes. An imbalance of immune cell function or discordance in cell orchestration at any stage can result in impaired wound healing. Tools to “fine tune” the immune response my lead to better treatment management and improve wound healing.

Chemokines are key players in the cellular orchestration that regulates wound healing. While they are involved in the stages of wound healing, chemokines are most abundant and varied during the inflammation and proliferation stages [5]. Chemokines recruit leukocytes, stimulate the activity of neutrophils, drive macrophage activity and polarization, and regulate the proliferation of fibroblasts and keratinocytes, which mediate collagen deposition [6]. Chemokines are also closely involved in angiogenesis, new vessel growth and extension [6]. There are approximately 20 chemokines that are known to be involved in wound healing, from across the four chemokine subfamilies (C, CC, CXC, and CX3C). Chemokines released from injured tissues bind to glycosaminoglycans (GAGs) on the surfaces of cells, in the vascular lumen glycocalyx or within extracellular matrices, where they can signal directly to cells, stimulating inter- and intra-cellular cascades, or drive chemotactic migration towards the site of injury [7]. Based on the understanding of the structure and function of these chemokines, new therapeutic methods to accelerate wound healing or to repair wounds with impaired healing by chemokine modulation can be developed [8,9]. These methods include chemokine depletion with biomaterials containing GAGs [10], immunotherapy with monoclonal antibodies against individual chemokines and chemokine receptors [9], or the topical application of recombinant chemokines [11]. The basis of some of these approaches remains incompletely understood.

The application of immune modulators from viruses as an approach toward deriving new protein therapeutics has been studied [12]. The co-evolution of viruses with their natural hosts invokes an adaptation arms race, whereby a successful strategy for the virus relies on immune evasion, often targeting key pathways that drive immune activation [13]. Large DNA viruses, such as poxviruses and herpesviruses, are adept at evading the innate immune system via a suite of virulence factors [14,15]. Translationally, these factors constitute a rich toolbox for developing immune modulators for treating disease [12].

Myxoma virus (MYXV) is a leporipoxvirus with well-known strict species-specificity and host-tropism to the European rabbit (Oryctolagus cuniculus) [16]. The safety and immunotherapeutic efficacy of several MYXV immune modulators in a wide array of preclinical models has been demonstrated [17-27]. M-T7 is an MYXV-derived immune modulator with broad chemokine-binding activity and therapeutic utility in inflammation-related diseases. M-T7 is expressed early in MYXV infection, and is the most abundantly secreted immune modulator [28]. In MYXV infection, M-T7 blocks lymphocyte infiltration into infected lesions by preventing chemokine gradient formation [29]. M-T7 is a soluble glycoprotein with the ability to directly bind all classes of chemokines (C, CC, CXC) tested in vitro, and decouple their interactions with GAG [30]. M-T7 treatment, given with or without concomitant cyclosporine, reduced acute renal transplant rejection, vasculopathy and scarring in rats [31]. M-T7 also markedly suppressed inflammatory cell invasion, and reduced acute and chronic aortic and renal transplant rejection, in mice in a manner dependent on heparan sulfation [23,32]. Thus, M-T7 can promote a resolution phenotype and a mechanism of tissue healing wherein the regulation of inflammation is critical. Here, we validate the use of recombinant M-T7 to modulate healing and local inflammatory cell responses at sites of full-thickness cutaneous wounds in a mouse model.

Materials and Methods

Recombinant M-T7 Production

Purified, recombinant M-T7 protein (m007L; NCBI Gene ID# 932081) was produced and provided by Viron Therapeutics (London, ON, Canada) and expressed and purified as previously described [33]. Briefly, the M-T7 coding sequence is inserted into pFastBacDual with a C-terminal His-tag and transformed into DH10Bac cells to generate bacmids as baculovirus shuttle vectors. Purified bacmids are transfected into Sf21 cells using

Cellfectin II reagent. Supernatants containing baculovirus are used to transduce High Five cells. Secreted M-T7 is purified by affinity tag purification over a Ni-NTA column with further purification by size exclusion chromatography via FPLC with a HiLoad 16/60 Superdex 75 column.

Animals

The animal procedures in this study were approved by the Institutional Animal Care and Use Committee of Arizona State University under protocol #17-1549R. Male and female wildtype C57BL6/J mice were bred on-site at Arizona State University. Mice aged 8-12 weeks were selected by simple randomization and used in this study. Mice were kept on a standard 12 h light-12 h dark cycle in a specific pathogen-free environment and given food and water ad libitum. Mice were single-housed after the wounding procedure to prevent interference with wound healing, as previously described [35].

Wounding Surgery and Measurement

We performed a splinted, full-thickness wound healing model as previously described [35]. In this model, a silicone splint is used to prohibit the wound contraction of mouse skin around a single, intrascapular full-thickness biopsy punch wound during the first seven days, effectively forcing second-intention healing as occurs in human skin (whereas mouse skin primary heals by contraction).

Briefly, mice were anesthetized by intraperitoneal injection of 0.1 mL per 25 g bodyweight of a cocktail of 120 mg/kg ketamine and 6 mg/kg xylazine. Once reaching the anesthetic surgical plane (as determined by toe pinch), mice were prepped by shaving a 1×1 inch area spanning from between the ears to the apex of the spine and centered between each shoulder. The shaved area was sterilized by two successive washes of 2% chlorhexidine gluconate solution (Dyna-Hex 2®, Xttrium Laboratories, Prospect, IL USA) followed by 70% ethanol with sterile cotton swabs. A small amount of veterinary ocular ointment was applied to each eye to prevent corneal drying. Mice were kept on a monitored heating pad for the duration of the procedure.

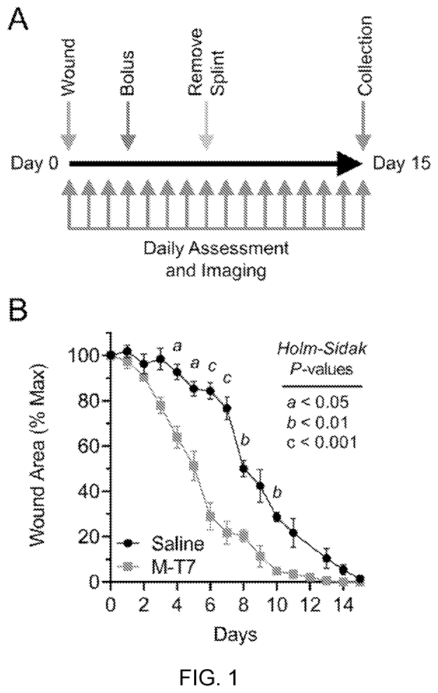

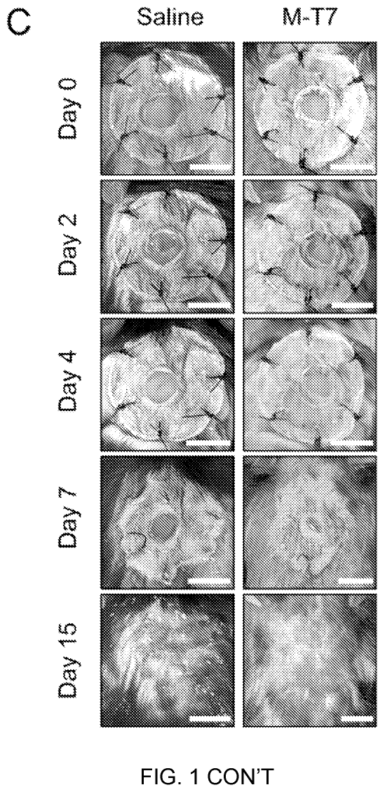

A full-thickness excisional wound was created with a 3.5 mm biopsy punch tool centered in the shaved area. Careful attention was paid to prevent damage to the panniculus carnosus beneath the skin. Immediately after creating the punch, the wounds were treated by application of 20 μL sterile normal 0.9% NaCl saline solution (N=17) or 20 μL sterile normal saline (0.9% NaCl) containing 1 μg recombinant M-T7 (N=16) applied directly to the wound bed with a micropipette. A donut-shaped silicon splint (O.D. 15 mm; I.D. 5.0 mm; Culture-Well™, Grace Biolabs, Bend, OR USA) with Tegaderm™ (3M Company, Saint Paul, MN USA) affixed to one side was coated with cyanoacrylate glue (Krazy Glue®) on the opposite side and carefully placed on the back of the mouse while keeping the wound centered within the inner diameter of the splint. Six interrupted sutures (4-0 black Ethilon monofilament with an FS-2 reverse cutting needle; Ethicon, Inc., Somerville, NJ USA) were placed around the outer circumference of the splint (approximately 2 mm inset from the edge) to complete the procedure. Mice were monitored on heating pads until awake and motile prior to returning to single-housed cages for the remainder of the experiment. On day 3 post-wounding, the mice were anesthetized with 1-3% isoflurane, to effect, and 20 μL saline or 20 saline containing 1 μg M-T7 was carefully applied topically to the wounds (drop-wise above the wound) by inserting an insulin syringe through the silicon splint, with care not to disrupt the healing wound bed during application. To prevent self-induced secondary skin damage from scratching, mice were again anesthetized with 1-3% isoflurane, to effect, on day 7 post-wounding and the splints were carefully removed with sterile surgical scissors before being returned to single-housed cages as previously described [35]. The experimental design is outlined in FIG. 1, panel A.

Wound Planimetry

Mice were assessed while awake on the day of the procedure (day 0) and on every subsequent day of follow-up for a total of 15 days. Digital images were collected along with a known size marker. Planimetric measurements of the wound healing progress were performed in ImageJ/FIJI and calibrated against the known size marker [36].

Immunohistochemistry and Herocivi's Polychrome Staining

Mice were euthanized in individual cohorts on days 2, 4, 7 and 15 post-wounding, and tissues were collected and fixed in 10% neutral-buffered formalin for 1 week before processing. Fixed tissues were processed and perfused with paraffin with a Leica TP1050 processor through graded alcohols and xylene, then embedded into paraffin cassettes on a Leica EG1160 embedding station. Blocks were sectioned using a Leica RIVI2165 microtome (5 μm sections) and stained with hematoxylin and eosin (H&E) according to standard procedures. Slides with sections that reached the wound site as determined by H&E screening were further stained by immunohistochemistry and collagen special staining.

Immunohistochemistry (IHC) was performed as previously described. Briefly, the slides were rehydrated through graded xylene and graded alcohols. Rehydrated slides were submerged in sodium citrate buffer, sandwiched with a clean glass slide to prevent tissue loss and incubated at 60° C. to retrieve epitopes. Endogenous peroxidases were quenched with 3% hydrogen peroxide in PBS and non-specific protein binding was blocked with 5% bovine serum albumin in TBS/0.1% Tween 20. Sections were probed overnight at 4° C. with rabbit polyclonal antibodies against Arginase-1 (Cell Signaling, #93668, 1:200), CD31 (Abcam, ab28364, 1:200), CD3 (Abeam, ab5690, 1:200) and CD4 (Abeam, ab183685, 1:1000), rabbit monoclonal antibodies against HSP47 (Abeam, ab109117, 1:300) or TGF-beta 1 (Abeam, ab215715, 1:500), or mouse monoclonal antibody against Ly6G (Invitrogen, #14-5931-82, 1:200). HRP-conjugated secondary antibodies against rabbit and mouse IgG (Jackson ImmunoResearch, West Grove, PA USA) were applied at a dilution of 1:500 for 1 to 2 h at room temperature. Antigens were revealed with ImmPACT DAB (Vector Labs, Burlingame, CA USA), counterstained with Gil's formula #3 Hematoxylin and mounted with Cytoseal XYL.

Herovici's Polychrome collagen stain kit was purchased from American MasterTech (Lodi, CA, USA). The slides were processed according to manufacturer's procedure and mounted with Cytoseal XYL.

Histopathology Imaging and Analysis

The slides were assessed on an Olympus BX51 upright microscope equipped with an Olympus DP74 CMOS high-resolution camera operated by cellSens Dimensions v1.16. Objective-calibrated TIFFs were analyzed and processed in ImageJ/FIJI. Positively stained cells were quantified per high power field using the Cell Counter plugin developed by Kurt De Vos and packaged with FIJI under a GPLv3 license. Herovici's Polychrome stains were quantified to produce a “Herovici Ratio” of pink stain (Type I collagen) versus blue stain (Type III collagen), where a higher ratio indicates more mature collagen and the less active deposition of immature collagen. Briefly, images were deconvoluted with the plugin Colour Deconvolution 1.7 using the methods described by Ruifrok and Johnson [37]. A region of interest was drawn in the dermis of the wound area and replicated to both red and blue channels. The integrated intensity (densitometry) of the region of interest was measured for each channel and the values were used to produce the Herovici Ratio.

ELISAs

Enzyme-linked immunosorbent assays (ELISA) were performed using Duo-Set kits for TNFα (DY410), VEGF (DY493) and CCL2 (DY479), from R&D Systems (Minneapolis, MN, USA). ELISAs were performed using tissues collected from individual cohorts of mice euthanized on days 1, 4 and 7. A 1 cm tissue sample centered on the wound was collected for each mouse, snap frozen and homogenized in RIPA lysis buffer according to manufacturer's procedures. Results were normalized to mg total protein as determined by BCA protein assay (Pierce, ThermoFisher Scientific, Carlbad, CA, USA).

Statistics

Analysis of statistical significance was performed by Two-Way Analysis of Variance (ANOVA) and Student's unpaired T-test using GraphPad Prism v8.2.1. The analyses passed normality tests according to Anderson—Darling (A2 *), D′Agostino—Pearson omnibus (K2), Shapiro—Wilk (W) and Kolmogorov—Smirnov (distance) with an alpha=0.05. P-values were considered significant at *p<0.05, **p<0.01, ***p<0.001 and ****p <0.0001, except in FIG. 1, panel B where a equates to p<0.05, b equates to p<0.01 and c equates to p<0.001 for the purposes of clarity in data presentation.

Results

Recombinant M-T7 promotes full-thickness wound healing

We analyzed the effects of recombinant M-T7 on the treatment of full-thickness wounds in a splinted wound healing model in wildtype C57BL6/J mice [35,38-40]. Based on effective doses and work with a second unrelated MYXV-derived immune modulating serine proteinase inhibitor, Serp-1, in the same model, we gave recombinant M-T7 topically in a dose of 1μg in 20 μL saline, with a second bolus of 1 μg in 20 μL saline 3 days post-wounding (FIG. 1, panel A) [35]. The control, saline-treated mice were similarly treated with a bolus of saline 3 days post-wounding. Daily planimetric measurements of wound healing progress demonstrate that M-T7 significantly accelerates full-thickness wound healing when given as a recombinant protein in a topical saline solution (FIG. 1, panels B,C). No evidence of wound site infection (pus, discharge, discoloration) was observed in the saline or M-T7-treated groups. We noted that during the first 8 days of healing (day 0 and up to 7 days post-wounding), healing was limited to only second-intention mechanisms due to the silicon splint preventing wound contraction (FIG. 1, panels C). This is a major benefit of the silicon splint model because, left on its own, mouse skin contracts within 1-2 days, which limits the interpretation of the healing process. By day 7, saline control-treated wounds had only achieved a mean of 23% closure, while M-T7 significantly accelerated the healing process and a 78% mean wound closure was achieved (p=0.0007). Even after removing the silicon splints, the wounds of M-T7-treated mice continued to close more quickly than mice treated with saline alone, and achieved full closure 3-4 days before saline-treated mice. Thus, recombinant M-T7 given during the early stage of healing has a sustained effect on accelerating wound closure.

Recombinant M-T7 Promotes Collagen Maturation in Wounds

Collagen deposition and maturation are key components of the wound healing process. The inappropriate deposition or impaired maturation of collagen are associated with

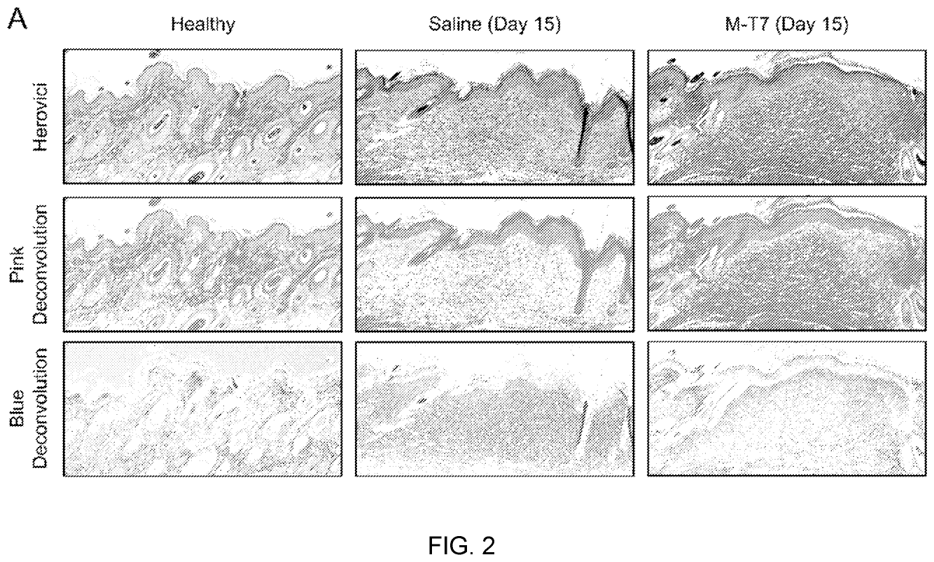

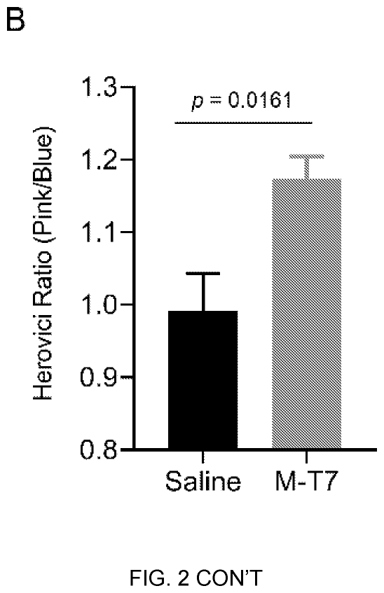

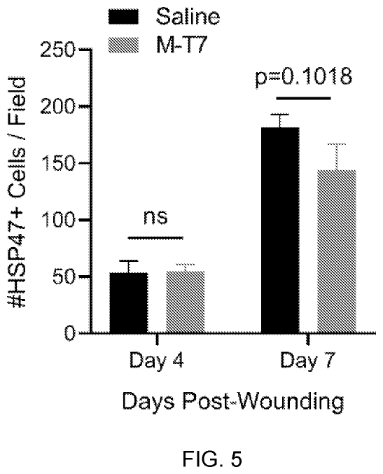

scarring and limited angiogenesis [41]. The improved remodeling of collagen in the healing wound can thus improve both scarring and healed tissue health via the promotion of improved angiogenesis. Herovici's polychrome is a histologic special stain which differentiates between immature Type III collagen (stained blue) and mature Type I collagen (stained pink) [42]. We used Herovici's polychrome to evaluate the collagen maturation of healed wounds after M-T7 treatment. Quantitative image analysis of the amount of pink Type I collagen staining versus the amount of blue Type III collagen showed what we refer to as the Herovici Ratio for the tissue. A higher Herovici Ratio (more pink, less blue) indicates more advanced collagen maturation, whereas a lower Herovici Ratio (less pink, more blue) indicates less collagen maturation and more active deposition of immature collagen. At day 15 post-wounding, the healed wounds of mice treated with M-T7 had significantly higher Herovici Ratios, indicating more mature Type I collagen than saline-treated mice (FIG. 2). Interestingly, this was not associated with an increase in fibroblast marker HSP47 (FIG. 5). These data indicate that, in addition to accelerated closure, wounds treated with M-T7 had a higher fidelity, healing with a more properly organized collagen architecture.

M-T7 Stimulates Peri-Wound Angiogenesis

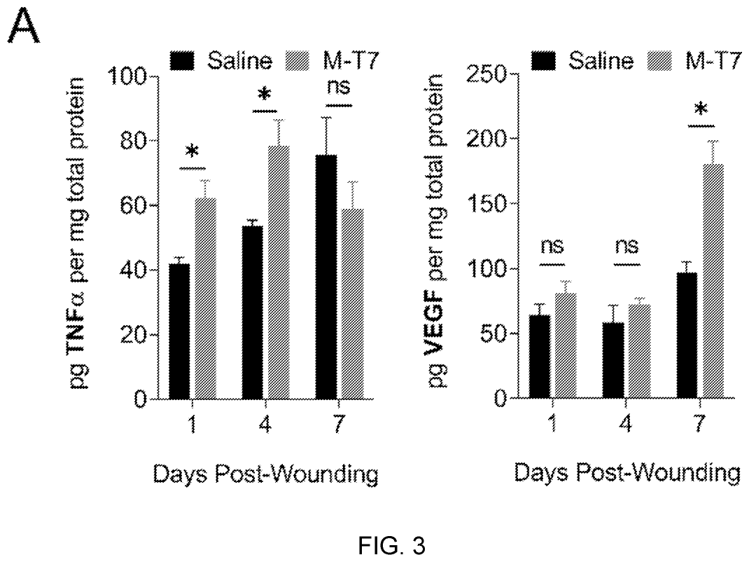

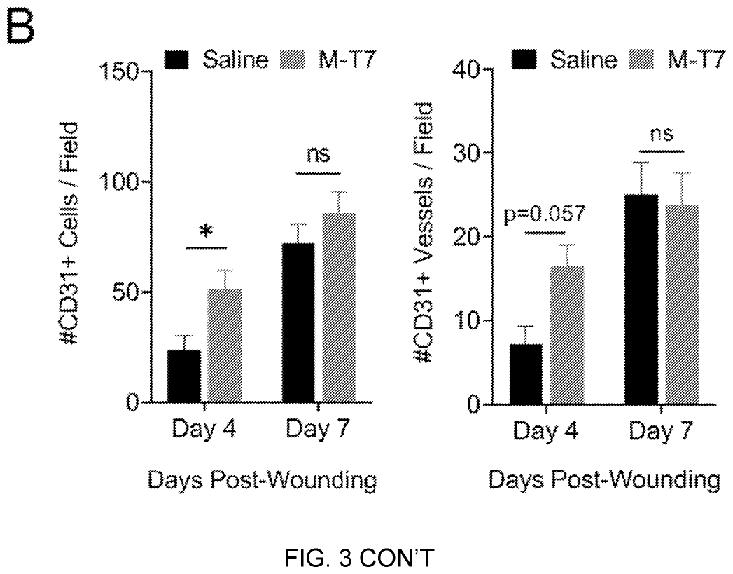

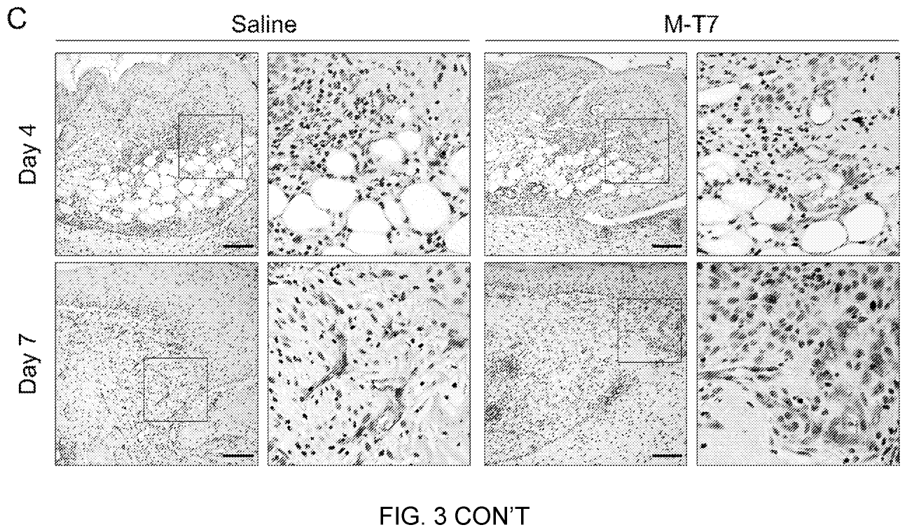

Angiogenesis is an essential component of the proliferative stage of cutaneous wound healing, characterized by an early and abundant burst of immature vessels which eventually regress into a mature vascular network via the activity of anti-angiogenic factors, such as Sprouty2 and PEDF [43]. Therapeutic strategies are now actively sought to enhance angiogenesis during wound healing [44]. Angiogenesis in the early stages of wound healing is driven by the priming of endothelial cells with pro-inflammatory cytokines, such as tumor necrosis factor alpha (TNFα), thereby inducing a tip cell phenotype [45]. In the context of tissue injury, TNFα is critical for the downstream production of vascular endothelial growth factor (VEGF), an essential growth factor in regulating angiogenesis [46]. We performed ELISA analyses of the healing wound's bed tissue on days 1, 4 and 7 post-wounding to quantitatively measure levels of TNFα and VEGF (FIG. 3, panel A). Recombinant M-T7 induced a significant increase in wound bed levels of TNFα on days 1 and 4 (p<0.05), and of VEGF by day 7 (p<0.05), versus saline treatment alone. We performed immunohistochemistry of wound tissues on days 4 and 7 post-wounding to determine the degree of angiogenesis by staining for CD31 (also called PECAM-1), a canonical marker for endothelial cells in the vasculature (FIG. 3, panels B, C). A quantification of the number of CD31+ cells and vessels per 20× field in the peri-wound area indicated a significant increase on day 4 post-wounding (p<0.05) versus saline treatment alone. We observed no significant difference on day 7 post-wounding. Qualitatively, we noted that the CD31+ cells formed more robust vessels in the wounds treated with M-T7, with increased length and thickness versus saline treatment alone (FIG. 3, panel C). Taken together, these results indicate that M-T7 stimulates an early TNFα response, which stimulates a more robust VEGF response, ultimately leading to accelerated angiogenesis in the peri-wound area associated with accelerated wound closure.

M-T7 Modulates Immune Responses in the Healing Wound

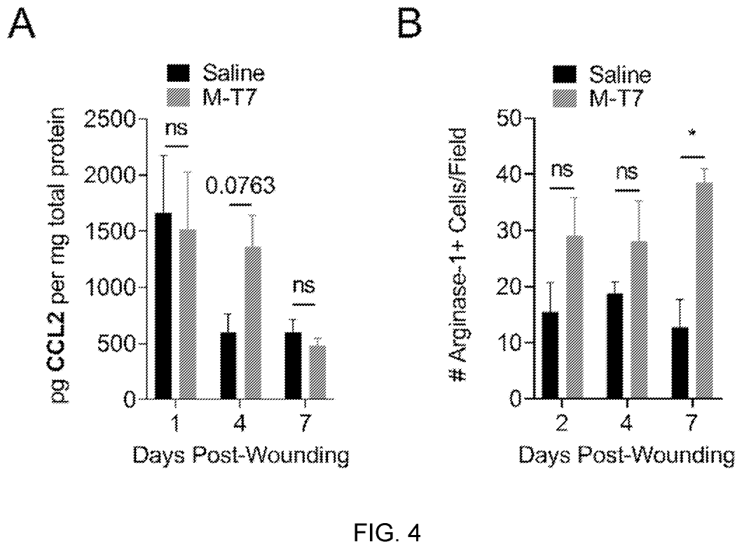

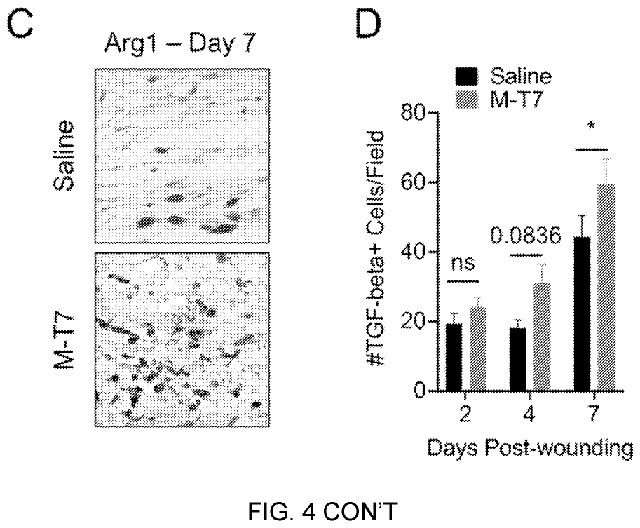

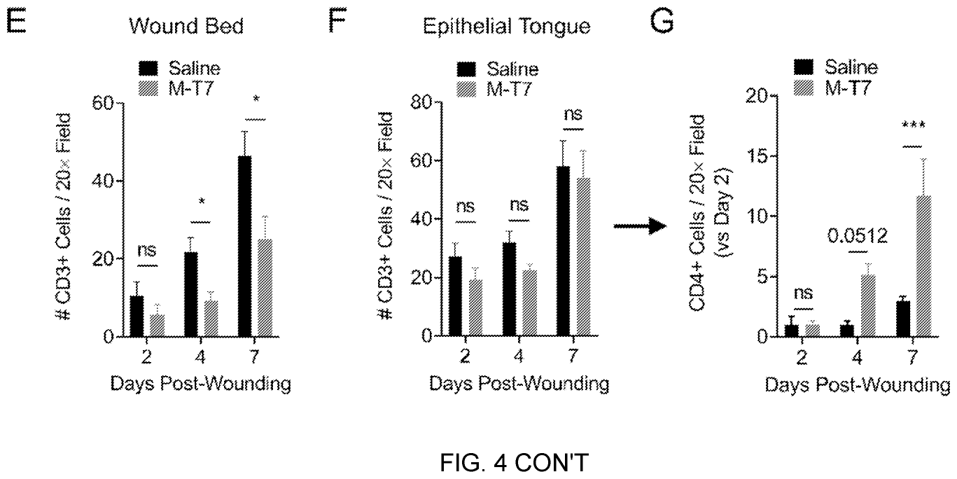

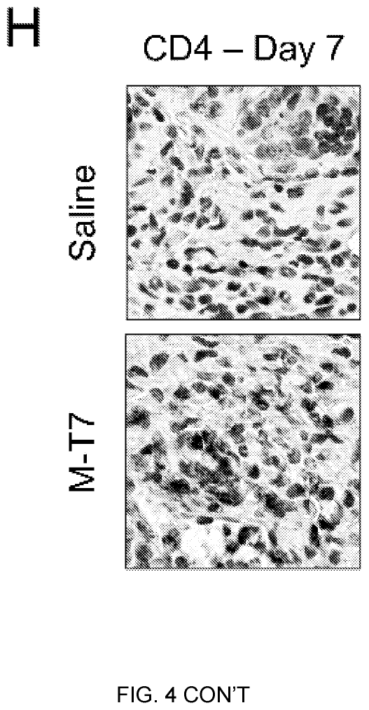

M-T7 binds to the classes of chemokines (C, CC and CXC) and inhibits their interaction with glycosaminoglycans, thereby preventing chemokine gradient formation [23,30,47]. CCL2, also called monocyte chemoattractant protein-1 (MCP-1), is a CC-class chemokine previously shown to have a critical role in the regulation of wound healing [48]. We performed the ELISA analysis of CCL2 on wound tissues treated with saline or M-T7, collected on days 1, 4 and 7 post-wounding (FIG. 4, panel A). Wounds treated with M-T7 had an elevated level of CCL2 on day 4 post-wounding, which approached significance (p=0.0763), while levels of CCL2 were not different between saline and M-T7 treatment on days 1 and 7 post-wounding. Independent of its chemotactic function, the signaling of CCL2 with its receptor, CCR2, was previously shown to promote the polarization of macrophages towards a pro-resolution (i.e., M2) phenotype [49]. We performed immunohistochemistry of wounds treated with saline or M-T7 on days 2, 4 and 7 post-wounding, staining for Arginase-1, a canonical marker of M2 macrophage polarization. The quantification of Arginase-1+ cells revealed a trend towards elevated M2 macrophages on days 2 and 4 post-wounding, which achieved significance (p<0.05) by day 7 post-wounding (FIG. 4, panels B,C). Accordingly, the number of TGF-beta+cells per field trended towards significance on day 4 (p=0.0836) and reached significance by day 7 post-wounding (p<0.05) (FIG. 4, panel D). We further validated the effects of M-T7 treatment on T cell infiltration in the healing wound. We found that M-T7 treatment significantly inhibited the infiltration of CD3+T cells, a general T cell marker, into the bed of the healing wound on days 4 and 7 post-wounding (FIG. 4, panel E), without inhibiting the accumulation of CD3+ cells in the epithelial tongue of the wounds (FIG. 4, panel F). Regulatory T cells, a CD4 T cell subtype, are crucial for the

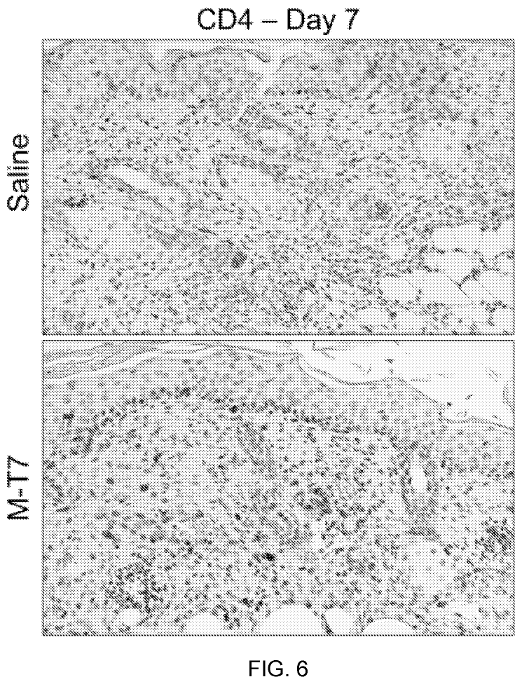

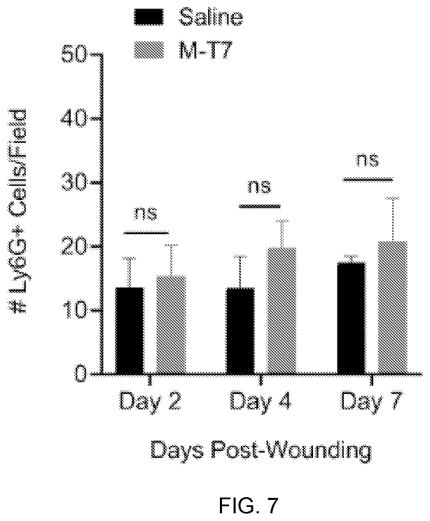

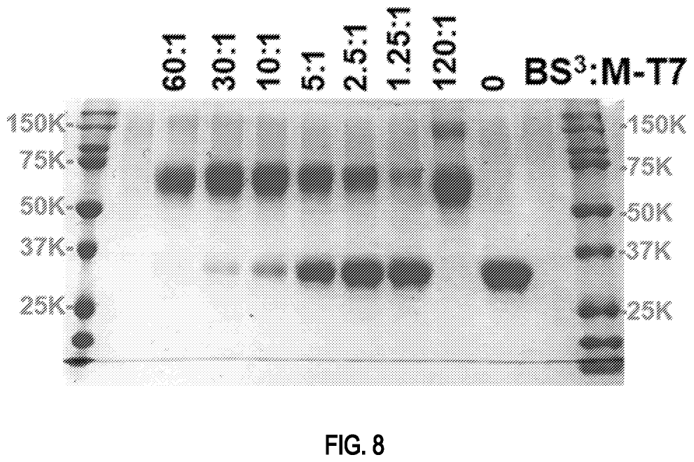

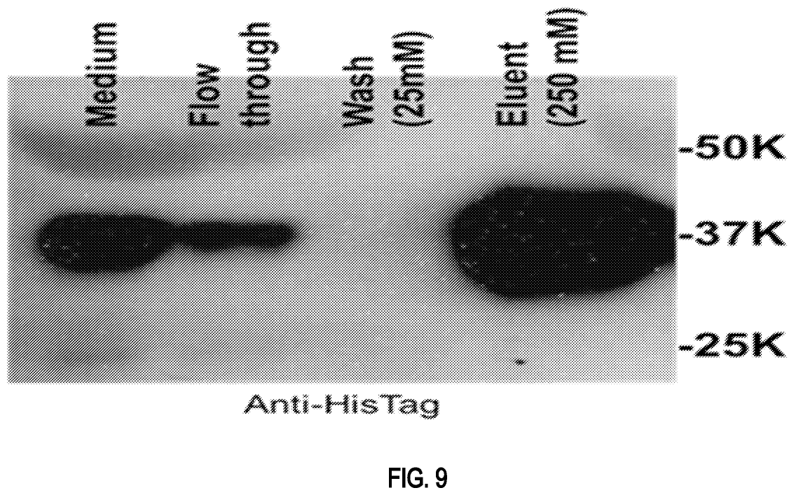

normal and accelerated healing of cutaneous wounds [50]. We found that M-T7 treatment significantly increased the accumulation of CD4+ cells in the epithelial tongue of healing wounds versus saline treatment alone (FIG. 4, panels G, H; FIG. 6). We did not observe an effect on neutrophil infiltration (FIG. 7). Thus, decoupling the chemokine-glycosaminoglycan gradient with M-T7 modulates the immune response in the wound environment to accelerate healing.

Discussion

Large cutaneous wounds, such as those associated poor healing (e.g., diabetic or aged), scarring and super-imposed infections, are a complex and costly medical burden, with an annual incidence of more than 6 million cutaneous wound cases and a collective yearly cost of more than USD 20 billion, not inclusive of the more than 170,000 scar revision

surgeries annually in the United States [51]. Comorbidities such as advanced age and diabetes, or complications such as infection, burns and battlefield conditions, further increase

the difficulty of wound management and the risk of adverse outcomes [52]. Investigation has thus intensified to address an unmet need for new treatments to accelerate wound healing.

In this study, we validated the therapeutic efficacy of recombinant Myxoma virus-derived immune modulating protein M-T7 in a mouse model of full-thickness wound healing. We administered two doses of recombinant M-T7 on days 0 and 3 post-wounding, mirroring the dosing regimen that we found to be optimal for another Myxoma virus-derived immune modulator, Serp-1, in a previous study [35]. Planimetric analysis revealed a significant acceleration of wound closure by treating wounds topically with M-T7. Acceleration occurred during the earliest stages of healing and was independent of contraction, as the silicone splints were not removed until day 7 post-wounding (FIG. 1, panel B). The first phase of healing is known to be a crucial period for protection against infection and the prevention of additional trauma as granulation tissue is formed [53].

A risk of accelerated wound healing is the deposition of disorganized connective tissue leading to scarring, such as in full-thickness skin wounds without contraction [54]. Druecke and colleagues investigated the use of different dermal regeneration templates on full-thickness wounds in a porcine model [55]. They found that while the Integra material, a composite scaffold of bovine hide collagen and shark chondroitin-6-sulfate, improved collagen maturation, there was slower tissue ingrowth, and tissue integrity was lost [55]. In contrast, the authors found that a bovine hide collagen sponge scaffold, produced via chemical crosslinking with 1-ethyl-3-(3-dimethylaminopropyl)carbodiimide (EDC), allowed more rapid ingrowth and overall tissue integrity, with no benefit to collagen maturation. Here, we found that in addition to accelerating wound healing, recombinant M-T7 also resulted in earlier, improved collagen maturation, as determined by quantification with Herovici's polychrome in a metric we term the “Herovici Ratio” (FIG. 2). A similar quantification of Type IILI collagen was observed by O'Rourke and colleagues in their investigation of accelerated wound healing by surfactant polymer dressings containing siRNA to Fidgetin-Like 2, with the authors noting increased red Type I collagen, illustrating higher-fidelity healing [56].

Angiogenesis is a critical component of wound healing, and several comorbidities associated with impaired wound healing, such as diabetes, also exhibit reduced angiogenesis [57,58]. Angiogenesis plays a critical role in providing the sufficient delivery of blood and nutrients, and access to reparative immune cells, during the course of healing. Accordingly, numerous groups have investigated approaches to accelerate healing by promoting angiogenesis. For example, recombinant TNFα applied directly to wounds accelerates the early stages of wound healing [59]. The therapeutic effect of TNFα in the early stage of wound healing can proceed via the induction of angiogenesis, as TNFα is a powerful inducer of VEGF and endothelial tip cell formation [45,46]. Other treatments shown to accelerate wound healing also act via the induction of angiogenesis. For example, topical simvastatin and asperosaponin VI both accelerate wound healing by enlisting the VEGF signaling cascade, and VEGF-C applied directly to wounds also accelerates healing [60-62]. Here, we show that a recombinant M-T7 treatment resulted in a significant increase in local TNFα during the earliest stages of wound healing, which temporally transitions into a significant increase in VEGF in the healing bed (FIG. 3, panel A). This coincides with the canonical, early inflammatory phase of healing, and the transition to the proliferation phase of healing

[63]. Accordingly, increased angiogenesis was directly observed by immunohistochemistry for CD31 in the peri-wound area (FIG. 3, panels B, C). Thus, the data indicates that topical M-T7 modulates the chemokine environment, resulting in the augmentation of pro-healing molecules in the wound environment, engaging the pro-angiogenesis signaling cascade at the level of both cytokines and growth factors, and resulting in a significant induction of angiogenesis at the boundaries of healing wounds associated with increased wound closure.

We sought to determine the effect of recombinant M-T7 on local immune responses in the healing wounds. Some virus-derived immune modulators, such as the herpesvirus M3 chemokine decoy receptor and Myxomavirus MT1, inhibit the ability of chemokines to signal to their receptor. M3 also uniquely blocks chemokine binding, both to GAGs and also to receptors [64]. In contrast, M-T7 acts primarily at the level of chemokine-glycosaminoglycan interactions [30,47,65,66]. M-T7 is an interferon gamma receptor homologue with specificity for rabbit interferon gamma [28]. The M-T7 inhibition of chemokine to GAG binding is found for the mammals tested to date, e.g., rabbits, rats, mice and human cells [30]. Thus, without wishing to be bound by theory, M-T7 treatment will inhibit chemokine gradient formation [47]. Indeed, M-T7 lost therapeutic efficacy in the absence of normal active heparan sulfation in mice with conditional endothelial deficiency of the heparan sulfotransferase enzyme Ndstl, with presumed consequences in modifying the formation of chemokine gradients [23]. CCL2/MCP-1 signaling is critical in regulating physiologic wound healing. For example, wounds made in mice deficient of CCL2 exhibit delayed re-epithelialization, reduced capillary density and impaired collagen remodeling [48]. In contrast, recombinant CCL2 treatment reverses impaired wound healing in diabetic mice by restoring macrophage responses [67,68]. Receptor engagement by chemokines induces the internalization of both the receptor and its ligand, resulting in intracellular degradation and recycling [69]. While CCL2 exists in dynamic equilibrium as both a monomer and dimer, only the monomeric form is capable of receptor engagement, and obligate dimeric mutants of CCL2 are incapable of signaling [70]. The dimerization of CCL2 requires glycosaminoglycan interactions [71]. We found increased levels of CCL2 in healing wounds when treated with recombinant M-T7 (FIG. 4, panel A), consistent with the role of CCL2 in improved healing. Without wishing to be bound by theory, M-T7 treatment inhibited the oligomerization of CCL2, slowing receptor engagement and delaying its subsequent degradation. Further, we found M-T7-dependent effects in two cell populations known to be affected by CCL2 and other chemokine signaling: macrophages and T cells. For example, we found an increase in M2-polarized, pro-resolution macrophages (FIG. 4, panel B). This is in agreement with work showing that CCL2 signaling results in M2 polarization [49]. We also found increased CD4+ T cells in the epithelial tongues of healing wounds treated with M-T7 (FIG. 4, panels F, G), in agreement with the ability for CCL2 to promote CD4 recruitment, and in the CD4-lineage cells driving accelerated wound healing [50,72]. CCL2 acts directly on T cells via the action of CCR2 and CCR4 [73], but can also induce the recruitment of CD4 cells into tissues in a promiscuous manner, using other receptors [72]. Further, M-T7 interacts with many chemokines, and can induce a broad milieu change in a range of chemokines. The specific targeted mechanisms of CD4 recruitment can be validated using genetic knockouts or neutralizing antibody treatments. Without wishing to be bound by theory, continued expression of CCL2 by local cells, combined with an off-rate of CCL2:M-T7 interactions, may have contributed to sustained CCL2 signaling. However, we cannot exclude the role of other chemokines or cytokines in the wound healing milieu in this study, as M-T7 interferes with GAG binding for C, CC and CXC chemokines in vitro. Thus, the decoupling of glycosaminoglycan interactions with chemokines by M-T7 has an effect on CCL2 signaling, and downstream effects on macrophage and T cell populations, leading to accelerated wound healing. The specific chemokine and GAG pathways modulated by M-T7 in the orchestration of local and infiltrating immune cells in the healing wound bed can be determined.

M-T7 is now the second Myxoma virus-derived immune modulator to exhibit efficacy in promoting wound healing. Serp-1, a serine protease inhibitor (serpin), is a glycosylated, secreted protein which targets serine proteases in both the thrombotic (FXa, thrombin) and thrombolytic (uPA, tPA, plasmin) cascades [20]. Recombinant Serp-1 accelerated full-thickness wound healing in mice [35]. In addition to the acceleration of wound closure, both Serp-1 and M-T7 resulted in an increase in VEGF and peri-wound angiogenesis, as well as an increase in pro-resolution M2-polarized macrophages. These findings underscore the benefits associated with developing therapeutics from virus-expressed immune modulators. These findings also again emphasize the safety of these immune modulators when used as therapeutics. First, the limited genomic space in a virus necessitates the evolution of multipotency (i.e., multiple targets of inhibition), providing highly potent and effective immune modulating molecules [74]. While Serp-1 targets numerous proteins in the clotting cascade, M-T7 targets the panoply of chemokines. Second, virus-derived immune modulators often exhibit potency at extremely low concentrations. Both Serp-1 and M-T7 function at doses of only 100 nanogram per gram bodyweight, the equivalent of microgram per kilogram dosing in humans; that is, the lowest end range for therapeutic biologics [75]. Indeed, Serp-1 has been shown to exhibit therapeutic efficacy down to the picogram per gram range [76]. Third, virus-derived immune modulators have undergone “research and development” in the evolutionary arms race between the virus and its host. In the case of Myxoma virus, an estimated 10 million years of evolution have gone into developing expert modulators of the host immune response [16]. Thus, the suite of immune modulating proteins in Myxoma virus are a valuable, highly optimized “medicine cabinet” for targeting immune-driven pathologies, and for harnessing immune function to enhance tissue repair [12].

Conclusions

We report here that treatment with recombinant M-T7, a Myxoma virus-derived chemokine signaling modulator, accelerates the rate of healing in full-thickness wounds in wildtype mice. M-T7 treatment improved connective tissue remodeling, and increased angiogenesis and pro-resolution immune cell phenotypes. The chemokine milieu of the healing wound bed is highly complex, and M-T7 can interact with classes of the C, CC and CXC chemokines. We observed an effect on CCL2, which was associated with effects on macrophages, T cells and endothelial cells. We will validate the precise mechanisms of M-T7's therapeutic effects on wound healing. Further, we will validate the effects of recombinant M-T7 in complex comorbidities such as diabetes, infection and burns, to develop next-generation versions of M-T7 with enhanced function, and to learn more about the fundamental role of chemokines in cutaneous wound healing. Thus, without wishing to be bound by theory, M-T7 represents a virus-derived therapeutic, a new class of protein biologics that can address the significant medical burden created by dermal wounds.

### REFERENCES CITED IN THIS EXAMPLE

1 Niu, Y.; Li, Q.; Ding, Y.; Dong, L.; Wang, C. Engineered delivery strategies for enhanced control of growth factor activities in wound healing. Adv. Drug Deliv. Rev. 2019, 146, 190-208.
2. Gurtner, G.C.; Werner, S.; Barrandon, Y.; Longaker, M.T. Wound repair and regeneration. Nature 2008, 453, 314-321.
3. Ellis, S.; Lin, E.J.; Tartar, D. Immunology of wound healing. Curr. Dermatol. Rep. 2018, 7, 350-358.
4. Larouche, J.; Sheoran, S.; Maruyama, K.; Martino, M.M. Immune regulation of skin wound healing: Mechanisms and novel therapeutic targets. Adv. Wound Care 2018, 7, 209-231.
5. Martins-Green, M.; Petreaca, M.; Wang, L. Chemokines and their receptors are key players in the orchestra that regulates wound healing. Adv. Wound Care 2013, 2, 327-347.
6. Ridiandries, A.; Tan, J.; Bursill, C. The role of chemokines in wound healing. Int. J. Mol. Sci. 2018, 19, 3217.
7. Yaron, J. R.; Kwiecien, J. M.; Zhang, L.; Ambadapadi, S.; Wakefield, D. N.; Clapp, W. L.; Dabrowski, W.; Burgin, M.; Munk, B. H.; McFadden, G.; et al. Modifying the organ matrix pre-engraftment: A new transplant paradigm? Trends Mol. Med. 2019, 626-639.
8. Satish, L. Chemokines as therapeutic targets to improve healing efficiency of chronic wounds. Adv. Wound Care 2015, 4, 651-659.
9. Rees, P. A.; Greaves, N. S.; Baguneid, M.; Bayat, A. Chemokines in wound healing and as potential therapeutic targets for reducing cutaneous scarring. Adv. Wound Care 2015, 4, 687-703.
10. Lohmann, N.; Schirmer, L.; Atallah, P.; Wandel, E.; Ferrer, R. A.; Werner, C.; Simon, J. C.; Franz, S.; Freudenberg, U. Glycosaminoglycan-based hydrogels capture chemokines and rescue wound healing deficiency. Sci. Transl. Med. 2017, 9, eaai9044.
11. Vågesjö, E.; Öhnstedt, E.; Mortier, A.; Lofton, H.; Huss, F.; Proost, P.; Roos, S.; Phillipson, M. Accelerated wound healing in mice by on-site production and delivery of CXCL12 by transformed lactic acid bacteria. Proc. Natl. Acad. Sci. USA 2018, 115, 1895-1900.
12. Yaron, J. R.; Zhang, L.; Guo, Q.; Burgin, M.; Schutz, L. N.; Awo, E.; Wise, L.; Krause, K. L.; Ildefonso, C. J.; Kwiecien, J. M.; et al. Deriving immune modulating drugs from viruses—A new class of biologics. J. Clin. Med. 2020, 9, 972.
13. Best, S. M.; Kerr, P. J. Coevolution of host and virus: The pathogenesis of virulent and attenuated strains of myxoma virus in resistant and susceptible European rabbits. Virology 2000, 267, 36-48.
14. Kerr, P.; McFadden, G. Immune responses to myxoma virus. Viral Immunol. 2002, 15, 229-246.
15. Spiesschaert, B.; McFadden, G.; Hermans, K.; Nauwynck, H.; van de Walle, G.R. The current status and future directions of myxoma virus, a master in immune evasion. Vet. Res. 2011, 42, 76.
16. Alves, J. M.; Carneiro, M.; Cheng, J. Y.; de Matos, A. L.; Rahman, M. M.; Loog, L.; Campos, P. F.; Wales, N.; Eriksson, A.; Manica, A.; et al. Parallel adaptation of rabbit populations to myxoma virus. Science 2019, 363, 1319-1326.
17. Liu, L.; Lalani, A.; Dai, E.; Seet, B.; Macauley, C.; Singh, R.; Fan, L.; McFadden, G.; Lucas, A. The viral anti-inflammatory chemokine-binding protein M-T7 reduces intimal hyperplasia after vascular injury. J. Clin. Invest. 2000, 105, 1613-1621.
18. Chen, H.; Zheng, D.; Abbott, J.; Liu, L.; Bartee, M.Y.; Long, M.; Davids, J.; Williams, J.; Feldmann, H.; Strong, J.; et al. Myxomavirus-derived serpin prolongs survival and reduces inflammation and hemorrhage in an unrelated lethal mouse viral infection. Antimicrob. Agents Chemother. 2013, 57, 4114-4127
19. Kwiecien, J. M.; Dabrowski, W.; Marzec-Kotarska, B.; Kwiecien-Delaney, C. J.; Yaron, J. R.; Zhang, L.; Schutz, L.; Lucas, A. R. Myxoma virus derived immune modulating proteins, M-T7 and Serp-1, reduce early inflammation after spinal cord injury in the rat model. Folia Neuropathol. 2019, 57, 41-50.
20. Mahon, B. P.; Ambadapadi, S.; Yaron, J. R.; Lomelino, C. L.; Pinard, M. A.; Keinan, S.; Kurnikov, I.; Macaulay, C.; Zhang, L.; Reeves, W.; et al. Crystal structure of cleaved serp-1, a myxomavirus-derived immune modulating serpin: Structural design of serpin reactive center loop peptides with improved therapeutic function. Biochemistry 2018, 57, 1096-1107.
21. Ambadapadi, S.; Munuswamy-Ramanuj am, G.; Zheng, D.; Sullivan, C.; Dai, E.; Morshed, S.; McFadden, B.; Feldman, E.; Pinard, M.; McKenna, R.; et al. Reactive Center Loop (RCL) peptides derived from serpins display independent coagulation and immune modulating activities. J. Biol. Chem. 2016, 291, 2874-2887.
22. Yaron, J. R.; Chen, H.; Ambadapadi, S.; Zhang, L.; Tafoya, A. M.; Munk, B. H.; Wakefield, D. N.; Fuentes, J.; Marques, B. J.; Harripersaud, K.; et al. Serp-2, a virus-derived apoptosis and inflammasome inhibitor, attenuates liver ischemia-reperfusion injury in mice. J. Inflamm. 2019, 16, 12.
23. Chen, H.; Ambadapadi, S.; Wakefield, D.; Bartee, M.; Yaron, J. R.; Zhang, L.; Archer-Hartmann, S. A.; Azadi, P.; Burgin, M.; Borges, C.; et al. Selective deletion of heparan sulfotransferase enzyme, Ndstl, in donor endothelial and myeloid precursor cells significantly decreases acute allograft rejection. Sci. Rep. 2018, 8, 13433.
24. Kwiecien, J. M.; Dabrowski, W.; Kwiecien-Delaney, B. J.; Kwiecien-Delaney, C. J.; Siwicka-Gieroba, D.; Yaron, J. R.; Zhang, L.; Delaney, K. H.; Lucas, A. R. Neuroprotective Effect of Subdural Infusion of Serp-1 in Spinal Cord Trauma. Biomedicines. 2020, 8, 372.
25. Bot, I.; von der Thüsen, J. H.; Donners, M. M. P. C.; Lucas, A.; Fekkes, M. L.; de Jager, S. C. A.; Kuiper, J.; Daemen, M. J. A. P.; van Berkel, T. J. C.; Heeneman, S.; et al. Serine protease inhibitor Serp-1 strongly impairs atherosclerotic lesion formation and induces a stable plaque phenotype in ApoE-/-mice. Circ. Res. 2003, 93, 464-471.
26. Viswanathan, K.; Bot, I.; Liu, L.; Dai, E.; Turner, P. C.; Togonu-Bickersteth, B.; Richardson, J.; Davids, J. A.; Williams, J. M.; Bartee, M. Y.; et al. Viral cross-class serpin inhibits vascular inflammation and T lymphocyte fratricide; A study in rodent models in vivo and human cell lines in vitro. PLoS ONE 2012, 7, e44694.
27. Dai, E.; Guan, H.; Liu, L.; Little, S.; McFadden, G.; Vaziri, S.; Cao, H.; Ivanova, I. A.; Bocksch, L.; Lucas, A. Serp-1, a viral anti-inflammatory serpin, regulates cellular serine proteinase and serpin responses to vascular injury. J. Biol. Chem. 2003, 278, 18563-18572.
28. Upton, C.; Mossman, K.; Mcfadden, G. Encoding of a Homolog of the IFN-y Receptor by Myxoma Virus. Science 1992, 258, 1369-1372.
29. Mossman, K.; Nation, P.; Macen, J.; Garbutt, M.; Lucas, A.; Mcfadden, G. Myxoma virus M-T7, a secreted homolog of the interferon-γ receptor, Is a critical virulence factor for the development of myxomatosis in European rabbits. Virology 1996, 215, 17-30.
30. Lalani, A. S.; Graham, K.; Mossman, K.; Raj arathnam, K.; Clark-Lewis, I.; Kelvin, D.; McFadden, G. The purified myxoma virus gamma interferon receptor homolog M-T7 interacts with the heparin-binding domains of chemokines. J. Virol. 1997, 71, 4356-4363.
31. Bedard, E. L. R. R.; Kim, P.; Jiang, J.; Parry, N.; Liu, L.; Wang, H.; Garcia, B.; Li, X.; McFadden, G.; Lucas, A.; et al. Chemokine-binding viral protein M-T7 prevents chronic rejection in rat renal allografts. Transplantation 2003, 76, 249-252.
32. Dai, E.; Liu, L. Y.; Wang, H.; Mclvor, D.; Sun, Y. M.; Macaulay, C.; King, E.; Munuswamy-Ramanuj am, G.; Bartee, M. Y.; Williams, J.; et al. Inhibition of Chemokine-Glycosaminoglycan interactions in donor tissue reduces mouse allograft vasculopathy and transplant rejection. PLoS ONE 2010, 5, e10510.
33. Bartee, M. Y.; Dai, E.; Liu, L.; Munuswamy-Ramanujam, G.; Macaulay, C.; McIvor, D.; McFadden, G.; Lucas, A. R. Chapter 10 M-T7. Measuring chemokine-modulating activity. Methods Enzymol. 2009, 460, 209-228.
34. Suresh, K. An overview of randomization techniques: An unbiased assessment of outcome in clinical research. J. Hum. Reprod. Sci. 2011, 4, 8-11.
35. Zhang, L.; Yaron, J. R.; Tafoya, A. M.; Wallace, S. E.; Kilbourne, J.; Haydel, S.; Rege, K.; McFadden, G.; Lucas, A. R. A virus-derived immune modulating serpin accelerates wound closure with improved collagen remodeling. J. Clin. Med. 2019, 8, 1626.
36. Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; et al. Fiji: An open-source platform for biological-image analysis. Nat. Methods 2012, 9, 676-682.
37. Ruifrok, A. C.; Johnston, D. A. Quantification of histochemical staining by color deconvolution. Anal. Quant. Cytol. Histol. 2001, 23, 291-299.
38. Galiano, R. D.; Michaels, V. J.; Dobryansky, M.; Levine, J. P.; Gurtner, G. C. Quantitative and reproducible murine model of excisional wound healing. Wound Repair Regen. 2004, 12, 485-492.
39. Wang, X.; Ge, J.; Tredget, E. E.; Wu, Y. The mouse excisional wound splinting model, including applications for stem cell transplantation. Nat. Protoc. 2013, 8, 302-309.
40. Dunn, L.; Prosser, H. C. G.; Tan, J. T. M.; Vanags, L. Z.; Ng, M. K. C.; Bursill, C. A. Murine model of wound healing. J. Vis. Exp. 2013, 75, 1-6.
41. Tonnesen, M. G.; Feng, X.; Clark, R. A. F. Angiogenesis in wound healing. J. Investig. Dermatol. Symp. Proc. 2000, 5, 40-46.
42. Levame, M.; Meyer, F. Herovici's picropolychromium. Application to the identification of type I and III collagens. Pathol. Biol. 1987, 35, 1183-1188.
43. DiPietro, L. A. Angiogenesis and wound repair: When enough is enough. J. Leukoc. Biol. 2016, 100, 979-984.
44. Veith, A. P.; Henderson, K.; Spencer, A.; Sligar, A. D.; Baker, A. B. Therapeutic strategies for enhancing angiogenesis in wound healing. Adv. Drug Deliv. Rev. 2019, 146, 97-125.
45. Sainson, R. C. A.; Johnston, D. A.; Chu, H. C.; Holderfield, M. T.; Nakatsu, M. N.; Crampton, S. P.; Davis, J.; Conn, E.; Hughes, C. C. W. TNF primes endothelial cells for angiogenic sprouting by inducing a tip cell phenotype. Blood 2008, 111, 4997-5007.
46. Ligresti, G.; Aplin, A. C.; Zorzi, P.; Morishita, A.; Nicosia, R. F. Macrophage-derived tumor necrosis factor-a is an early component of the molecular cascade leading to angiogenesis in response to aortic injury. Arterioscler. Thromb. Vasc. Biol. 2011, 31, 1151-1159.
47. Bartee, M. Y.; Chen, H.; Dai, E.; Liu, L. Y.; Davids, J. A.; Lucas, A. Defining the anti-inflammatory activity of a potent myxomaviral chemokine modulating protein, M-T7, through site directed mutagenesis. Cytokine 2014, 65, 79-87.
48. Low, Q. E. H.; Drugea, I. A.; Duffner, L. A.; Quinn, D. G.; Cook, D. N.; Rollins, B. J.; Kovacs, E. J.; DiPietro, L. A. Wound healing in MIP-1α-/- and MCP-1-/- mice. Am. J. Pathol. 2001, 159, 457-463.
49. Sierra-Filardi, E.; Nieto, C.; Dominguez-Soto, Â.; Barroso, R.; Sânchez-Mateos, P.; Puig-Kroger, A.; Lopez-Bravo, M.; Joven, J.; Ardavin, C.; Rodriguez-Fernandez, J. L.; et al. CCL2 shapes macrophage polarization by GM-CSF and M-CSF: Identification of CCL2/CCR2-dependent gene expression profile. J. Immunol. 2014, 192, 3858-3867.
50. Nosbaum, A.; Prevel, N.; Truong, H.-A.; Mehta, P.; Ettinger, M.; Scharschmidt, T. C.; Ali, N. H.; Pauli, M. L.; Abbas, A. K.; Rosenblum, M. D. Cutting edge: Regulatory T cells facilitate cutaneous wound healing. J. Immunol. 2016, 196, 2010-2014.
51. Lim, A. F.; Weintraub, J.; Kaplan, E. N.; Januszyk, M.; Cowley, C.; McLaughlin, P.; Beasley, B.; Gurtner, G. C.; Longaker, M. T. The embrace device significantly decreases scarring following scar revision surgery in a randomized controlled trial. Plast. Reconstr. Surg. 2014, 133, 398-405.
52. Beyene, R. T.; Derryberry, S. L.; Barbul, A. The effect of comorbidities on wound healing. Surg. Clin. North Am. 2020, 100, 695-705.
53. Landên, N. X.; Li, D.; Stable, M. Transition from inflammation to proliferation: A critical step during wound healing. Cell. Mol. Life Sci. 2016, 73, 3861-3885.
54. De Vries, H.; Zeegelaar, J.; Middelkoop, E.; Gijsbers, G.; Van Marle, J.; Wildevuur, C.; Westerhof, W. Reduced wound contraction and scar formation in punch biopsy wounds. Native collagen dermal substitutes. A clinical study. Br. J. Dermatol. 1995, 132, 690-697.
55. Druecke, D.; Lamme, E. N.; Hermann, S.; Pieper, J.; May, P. S.; Steinau, H. U.; Steinstraesser, L. Modulation of scar tissue formation using different dermal regeneration templates in the treatment of experimental full-thickness wounds. Wound Repair Regen. 2004, 12, 518-527.
56. O'Rourke, B. P.; Kramer, A. H.; Cao, L. L.; Inayathullah, M.; Guzik, H.; Raj adas, J.; Nosanchuk, J. D.; Sharp, D. J. Fidgetin-like 2 siRNA enhances the wound healing capability of a surfactant polymer dressing. Adv. Wound Care 2019, 8, 91-100.
57. Okonkwo, U. A.; Chen, L.; Ma, D.; Haywood, V. A.; Barakat, M.; Urao, N.; DiPietro, L. A. Compromised angiogenesis and vascular Integrity in impaired diabetic wound healing. PLoS ONE 2020, 15, e0231962.
58. Lerman, O. Z.; Galiano, R. D.; Armour, M.; Levine, J. P.; Gurtner, G. C. Cellular dysfunction in the diabetic fibroblast: Impairment in migration, vascular endothelial growth factor production, and response to hypoxia. Am. J. Pathol. 2003, 162, 303-312.
59. Ritsu, M.; Kawakami, K.; Kanno, E.; Tanno, H.; Ishii, K.; Imai, Y.; Maruyama, R.; Tachi, M. Critical role of tumor necrosis factor-a in the early process of wound healing in skin. J. Dermatol. Dermatol. Surg. 2017, 21, 14-19.
60. Wang, C. G.; Lou, Y. T.; Tong, M. J.; Zhang, L. L.; Zhang, Z. J.; Feng, Y. Z.; Li, S.; Xu, H. Z.; Mao, C. Asperosaponin VI promotes angiogenesis and accelerates wound healing in rats via up-regulating HIF-la/VEGF signaling. Acta Pharmacol. Sin. 2018, 39, 393-404.
61. Saaristo, A.; Tammela, T.; Farkkila, A.; Kärkkäinen, M.; Suominen, E.; Yla-Herttuala, S.; Alitalo, K. Vascular endothelial growth factor-C accelerates diabetic wound healing. Am. J. Pathol. 2006, 169, 1080-1087.
62. Asai, J.; Takenaka, H.; Hirakawa, S.; Sakabe, J. I.; Hagura, A.; Kishimoto, S.; Maruyama, K.; Kajiya, K.; Kinoshita, S.; Tokura, Y.; et al. Topical simvastatin accelerates wound healing in diabetes by enhancing angiogenesis and lymphangiogenesis. Am. J. Pathol. 2012, 181, 2217-2224.
63. Singer, A. J.; Clark, R. A. F. Cutaneous wound healing. N. Engl. J. Med. 1999, 341, 738-746.
64. Millward, J. M.; Holst, P. J.; Høgh-Petersen, M.; Thomsen, A. R.; Christensen, J. P.; Owens, T. The murine gammaherpesvirus-68 chemokine-binding protein M3 inhibits experimental autoimmune encephalomyelitis. J. Neuroimmunol. 2010, 224, 45-50.
65. Parry, C. M.; Simas, J. P.; Smith, V. P.; Stewart, C. A.; Minson, A. C.; Efstathiou, S.; Alcami, A. A broad spectrum secreted chemokine binding protein encoded by a herpesvirus. J. Exp. Med. 2000, 191, 573-578.
66. Alexander, J. M.; Nelson, C. A.; van Berkel, V.; Lau, E. K.; Studts, J. M.; Brett, T. J.; Speck, S. H.; Handel, T. M.; Virgin, H. W.; Fremont, D. H. Structural basis of chemokine sequestration by a herpesvirus decoy receptor. Cell 2002, 111, 343-356.
67. Ishida, Y.; Kuninaka, Y.; Nosaka, M.; Furuta, M.; Kimura, A.; Taruya, A.; Yamamoto, H.; Shimada, E.; Akiyama, M.; Mukaida, N.; et al. CCL2-mediated reversal of impaired skin wound healing in diabetic mice by normalization of neovascularization and collagen accumulation. J. Invest. Dermatol. 2019, 139, 2517-2527.
68. Wood, S.; Jayaraman, V.; Huelsmann, E. J.; Bonish, B.; Burgad, D.; Sivaramakrishnan, G.; Qin, S.; DiPietro, L. A.; Zloza, A.; Zhang, C.; et al. Pro-inflammatory chemokine CCL2 (MCP-1) promotes healing in diabetic wounds by restoring the macrophage response. PLoS ONE 2014, 9, e91574.
69. Stone, M. J.; Hayward, J. A.; Huang, C.; Huma, Z. E.; Sanchez, J. Mechanisms of regulation of the chemokine-receptor network. Int. J. Mol. Sci. 2017, 18, 342, ISBN 6139902924.
70. Tan, J. H. Y.; Canals, M.; Ludeman, J. P.; Wedderburn, J.; Boston, C.; Butler, S. J.; Carrick, A. M.; Parody, T. R.; Taleski, D.; Christopoulos, A.; et al. Design and receptor interactions of obligate dimeric mutant of chemokine monocyte chemoattractant protein-1 (MCP-1). J. Biol. Chem. 2012, 287, 14692-14702.
71. Lau, E. K.; Paavola, C. D.; Johnson, Z.; Gaudry, J. P.; Geretti, E.; Borlat, F.; Kungl, A. J.; Proudfoot, A. E.; Handel, T. M. Identification of the glycosaminoglycan binding site of the CC chemokine, MCP-1: Implications for structure and function in vivo. J. Biol. Chem. 2004, 279, 22294-22305.
72. Cédile, 0. ; Wlodarczyk, A.; Owens, T. CCL2 recruits T cells into the brain in a CCR2-independent manner. Apmis 2017, 125, 945-956.
73. Bakos, E.; Thaiss, C. A.; Kramer, M. P.; Cohen, S.; Radomir, L.; Orr, I.; Kaushansky, N.; Ben-Nun, A.; Becker-Herman, S.; Shachar, I. CCR2 regulates the immune response by modulating the interconversion and function of effector and regulatory t cells. J. Immunol. 2017, 198, 4659-4671.
74. Finlay, B. B.; McFadden, G. Anti-immunology: Evasion of the host immune system by bacterial and viral pathogens. Cell 2006, 124, 767-782.
75. Tardif, J. -C.; L'Allier, P. L.; Grêgoire, J.; Ibrahim, R.; McFadden, G.; Kostuk, W.; Knudtson, M.; Labinaz, M.; Waksman, R.; Pepine, C. J.; et al. A randomized controlled, phase 2 trial of the viral serpin Serp-1 in patients with acute coronary syndromes undergoing percutaneous coronary intervention. Circ. Cardiovasc. Interv. 2010, 3, 543-548.
76. Lucas, A.; Liu, L. -y.; Macen, J.; Nash, P.; Dai, E.; Stewart, M.; Graham, K.; Etches, W.; Boshkov, L.; Nation, P. N.; et al. Virus-encoded serine proteinase inhibitor SERP-1 inhibits atherosclerotic plaque development after balloon angioplasty. Circulation 1996, 94, 2890-2900.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A topical formulation for promoting wound healing, the formulation comprising a therapeutically effective amount of an M-T7 polypeptide.

2. The topical formulation of claim 1, further comprising a pharmaceutically acceptable carrier.

3. The topical formulation of claim 1, wherein the M-T7 polypeptide comprises a biologically active fragment of M-T7 derived from Myxoma-virus.

4. The topical formulation of claim 1, wherein the M-T7 polypeptide is recombinantly produced.

5. The topical formulation of claim 1, wherein the M-T7 polypeptide has an amino acid sequence at least 80% identical to the amino acid sequence of
(SEQ ID NO: 1)
MDGRLVFLLASLAIVSDAVRLTSYDLNTFVTWQDDGYTYNVSIKPYTTA
TWINVCEWASSSCNVSLALQYDLDVVSWARLTRVGKYTEYSLEPTCAVA
RFSPPEVQLVRTGTSVEVLVRHPVVYLRGQEVSVYGHSFCDYDFGYKTI
FLFSKNKRAEYVVPGRYCDNVECRFSIDSQESVCATAVLTYGDSYRSEA
GVEVCVPELAKREVSPYIVKKSSDLEYVKRAIHNEYRLDTSSEGRRLEE
LYLTVASMFERLVEDVFE

6. The topical formulation of claim 1, wherein the M-T7 polypeptide has an amino acid sequence at least 80% identical to the amino acid sequence of
(SEQ ID NO: 2)
VRLTSYDLNTFVTWQDDGYTYNVSIKPYTTATWINVCEWASSSCNVSLA
LQYDLDVVSWARLTRVGKYTEYSLEPTCAVARFSPPEVQLVRTGTSVEV
LVRHPVVYLRGQEVSVYGHSFCDYDFGYKTIFLFSKNKRAEYVVPGRYC
DNVECRFSIDSQESVCATAVLTYGDSYRSEAGVEVCVPELAKREVSPYI
VKKSSDLEYVKRAIHNEYRLDTSSEGRRLEELYLTVASMFERLVEDVFE

7. The topical formulation of claim 1, wherein the M-T7 polypeptide lacks the N-terminus secretion sequence.

8. The topical formulation of claim 5 or claim 6, wherein the M-T7 polypeptide has a minimal amino acid sequence at least 80% identical to a polypeptide fragment within SEQ ID NO: 1 or SEQ ID NO: 2.

9. The topical formulation of claim 1, further comprising one or more additional active ingredients.

10. The topical formulation of claim 9, wherein the one or more additional active ingredients comprises an antibiotic, a pain reliever, an anti-inflammatory, an anti-scarring agent, a moisturizer, a steroid, an immune modulator, or a growth factor.

11. The topical formulation of claim 1, wherein the topical formulation is contained in a hydrophilic polymer.

12. The topical formulation of claim 11, wherein the hydrophilic polymer comprises a hydrogel.

13. The topical formulation of claim 12, wherein the hydrogel comprises chitosan, collagen, glycerine, aloe vera, methyl paraben, hydrogenated castor oil, hyaluronic acid, polypeptides, pHEMA, pHPMA, or any combination thereof.

14. The topical formulation of claim 1, wherein the M-T7 polypeptide comprises one or more post-translational modifications.

15. The topical formulation of claim 1, wherein the M-T7 polypeptide comprises one or more modifications to a post-translational modification.

16. The topical formulation of claim 14 or 15, wherein the post-translational modification is selected from the group consisting of PEGylation, sialylated, glycosylation, acetylation, acylation, lipid modification, palmitoylation, palmitate addition, phosphorylation, or glycolipid modification.

17. The topical formulation of claim 1, wherein the M-T7 polypeptide comprises at least one mutation.

18. The topical formulation of claim 1, wherein the composition is formulated as a topical ointment, cream, lotion, suspension, aqueous solution, dispersion, salve, gel, spray, or paste.

19. A nucleic acid encoding the M-T7 polypeptide of any one of claims 1-18. The nucleic acid of claim 19, wherein the nucleic acid has a nucleic acid sequence at least 80% identical to the nucleic acid of claim 19.

21. A vector comprising the nucleic acid of claim 16.

22. A cell comprising the vector of claim 18.

23. A wound dressing or bandage comprising a therapeutically effective amount of an M-T7 polypeptide of claim 1, wherein the polypeptide comprises a formulation that is added to, coated, on, or embedded into the wound dressing or bandage.

24. A method of treating a wound in a subject in need thereof, the method comprising administering topically onto the wound the topical formulation of any one of claims 1-15 or the wound dressing of claim 20 to the wound in the subject.

25. The method of claim 20, wherein the wound is a dermal wound, a chronic wound, an infected wound, a burn wound, a diabetic wound, a skin wound, or a cutaneous wound.

26. A method of promoting angiogenesis in a subject in need thereof, the method comprising administering topically onto the wound the topical formulation of any one of claims 1-15 or the wound dressing of claim 20 to the wound in the subject.

27. A kit comprising the topical formulation according to claim 1.
